SEQUENCE LISTING (54 sequences)

SEQ ID NO: 1 - DNA, 13 bp, Human immunodeficiency virus
taactagggaacc

SEQ ID NO: 2 - DNA, 8 bp, Human immunodeficiency virus
cactgctt

SEQ ID NO: 3 - DNA, 13 bp, Human immunodeficiency virus
aagcctcaataaa

SEQ ID NO: 4 - DNA, 34 bp, Human immunodeficiency virus
taactagggaacccactgcttaagcctcaataaa

SEQ ID NO: 5 - DNA, 34 bp, Human immunodeficiency virus
taactagggaaccgcatacatggttccctagtta

SEQ ID NO: 6 - DNA, 34 bp, Human immunodeficiency virus
taactagggaacccactgcttggttccctagtta

SEQ ID NO: 7 - DNA, 34 bp, Human immunodeficiency virus
tttattgaggcttgcatacataagcctcaataaa

SEQ ID NO: 8 - DNA, 34 bp, Human immunodeficiency virus
tttattgaggcttcactgcttaagcctcaataaa

SEQ ID NO: 9 - DNA, 34 bp, Human immunodeficiency virus
acccactgcttaagcctcaataaagcttgccttg

SEQ ID NO: 10 - DNA, 34 bp, Human immunodeficiency virus
ctgcttaagcctcaataaagcttgccttgagtgc

SEQ ID NO: 11 - DNA, 34 bp, Bacteriophage P1
ataacttcgtatagcatacattatacgaagttat

SEQ ID NO: 12 - DNA, 34 bp, Human immunodeficiency virus
  misc_feature (14)..(21): n is a, c, g, or t
taactagggaaccnnnnnnnnggttccctagtta

SEQ ID NO: 13 - DNA, 34 bp, Human immunodeficiency virus
  misc_feature (14)..(21): n is a, c, g, or t
tttattgaggcttnnnnnnnnaagcctcaataaa

SEQ ID NO: 14 - DNA, 34 bp, Human immunodeficiency virus
  misc_feature (14)..(21): n is a, c, g, or t
acccactgcttaannnnnnnnaaagcttgccttg

SEQ ID NO: 15 - DNA, 34 bp, Human immunodeficiency virus
  misc_feature (14)..(21): n is a, c, g, or t
ctgcttaagcctcnnnnnnnnttgccttgagtgc

SEQ ID NO: 16 - DNA, 34 bp, Human immunodeficiency virus
ataacttcgaaccgcatacatggttcgaagttat

SEQ ID NO: 17 - DNA, 34 bp, Human immunodeficiency virus
ataacttcggcttgcatacataagccgaagttat

SEQ ID NO: 18 - DNA, 13 bp, Human immunodeficiency virus
acccactgcttaa

SEQ ID NO: 19 - DNA, 8 bp, Human immunodeficiency virus
gcctcaat

SEQ ID NO: 20 - DNA, 13 bp, Human immunodeficiency virus
aaagcttgccttg

SEQ ID NO: 21 - DNA, 13 bp, Human immunodeficiency virus
ctgcttaagcctc

SEQ ID NO: 22 - DNA, 8 bp, Human immunodeficiency virus
aataaagc

SEQ ID NO: 23 - DNA, 13 bp, Human immunodeficiency virus
ttgccttgagtgc

SEQ ID NO: 24 - DNA, 50 bp, Artificial Sequence
  primer
tcgagctctgtacaaggaggaattcaccatgtccaatttactgaccgtac

SEQ ID NO: 25 - DNA, 29 bp, Artificial Sequence
  primer
ctctagactaatcgccatcttccagcagg

SEQ ID NO: 26 - DNA, 22 bp, Artificial Sequence
  primer
cttcgctattacgccagctggc

SEQ ID NO: 27 - DNA, 20 bp, Artificial Sequence
  primer
cactttatgcttccggctcg

SEQ ID NO: 28 - DNA, 21 bp, Human immunodeficiency virus
taactagggaacccactgctt

SEQ ID NO: 29 - DNA, 21 bp, Human immunodeficiency virus
cactgcttaagcctcaataaa

SEQ ID NO: 30 - DNA, 5356 bp, Artificial Sequence
  recombinant plasmid
gctagcgaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgg

SEQ ID NO: 31 - DNA, 34 bp, Human immunodeficiency virus
  misc_feature (14)..(21): n is a, c, g, or t
taactagggaaccnnnnnnnnaagcctcaataaa

SEQ ID NO: 32 - DNA, 34 bp, Human immunodeficiency virus
ataacttggaaccgcatacatggttccaagttat

SEQ ID NO: 33 - DNA, 34 bp, Human immunodeficiency virus
ataacttaggcttgcatacataagcctaagttat

SEQ ID NO: 34 - DNA, 34 bp, Human immunodeficiency virus
ataacttcgtatacactgctttatacgaagttat

SEQ ID NO: 35 - DNA, 34 bp, Human immunodeficiency virus
ataacagggtatagcatacattataccctgttat

SEQ ID NO: 36 - DNA, 34 bp, Human immunodeficiency virus
ataactgagtatagcatacattatactcagttat

SEQ ID NO: 37 - DNA, 34 bp, Human immunodeficiency virus
taactagcgtatagcatacattatacgctagtta

SEQ ID NO: 38 - DNA, 34 bp, Human immunodeficiency virus
tttattgcgtatagcatacattatacgcaataaa

SEQ ID NO: 39 - DNA, 34 bp, Human immunodeficiency virus
taactttcgtatagcatacattatacgaaagtta

SEQ ID NO: 40 - DNA, 34 bp, Human immunodeficiency virus
  misc_feature (14)..(21): n is a, c, g, or t
acccactgcttaannnnnnnnttaagcagtgggt

SEQ ID NO: 41 - DNA, 34 bp, Human immunodeficiency virus
acccactgcttaagcctcaatttaagcagtgggt

SEQ ID NO: 42 - DNA, 34 bp, Human immunodeficiency virus
acccactgcttaagcatacatttaagcagtgggt

SEQ ID NO: 43 - DNA, 34 bp, Human immunodeficiency virus
  misc_feature (14)..(21): n is a, c, g, or t
caaggcaagctttnnnnnnnnaaagcttgccttg

SEQ ID NO: 44 - DNA, 34 bp, Human immunodeficiency virus
caaggcaagctttgcctcaataaagcttgccttg

SEQ ID NO: 45 - DNA, 34 bp, Human immunodeficiency virus
caaggcaagctttgcatacataaagcttgccttg

SEQ ID NO: 46 - DNA, 34 bp, Human immunodeficiency virus
  misc_feature (14)..(21): n is a, c, g, or t
ctgcttaagcctcnnnnnnnngaggcttaagcag

SEQ ID NO: 47 - DNA, 34 bp, Human immunodeficiency virus
ctgcttaagcctcaataaagcgaggcttaagcag

SEQ ID NO: 48 - DNA, 34 bp, Human immunodeficiency virus
ctgcttaagcctcgcatacatgaggcttaagcag

SEQ ID NO: 49 - DNA, 34 bp, Human immunodeficiency virus
  misc_feature (14)..(21): n is a, c, g, or t
gcactcaaggcaannnnnnnnttgccttgagtgc

SEQ ID NO: 50 - DNA, 34 bp, Human immunodeficiency virus
gcactcaaggcaaaataaagcttgccttgagtgc

SEQ ID NO: 51 - DNA, 34 bp, Human immunodeficiency virus
gcactcaaggcaagcatacatttgccttgagtgc

SEQ ID NO: 52 - DNA, 34 bp, Human immunodeficiency virus
ctgcttaagcctcgcatacatttgccttgagtgc

SEQ ID NO: 53 - DNA, 34 bp, Human immunodeficiency virus
acccactgcttaagcatacataaagcttgccttg

SEQ ID NO: 54 - DNA, 34 bp, Human immunodeficiency virus
taactagggaaccgcatacataagcctcaataaa

(19) United States
(12) Patent Application Publication
(10) Pub. No.: US 2010/0285464 A1
(43) Pub. Date: Nov. 11, 2010

(54) A CONSERVED REGION OF THE HIV-1 GENOME AND USES THEREOF

(75) Inventors: Nir CARMI, Ramat Efal (IL); Noa MATARASSO, Ramat Aviv (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignees: RECOGENE LTD, Tel Aviv (IL); State of Israel Ministry of Agriculture Agricultural Research Organization, Bet Dagan (IL)

(21) Appl. No.: 12/668,437

(22) PCT Filed: Jul. 13, 2008
(86) PCT No.: PCT/IL2008/000968

§ 371 Date: Jun. 1, 2010

Related U.S. Application Data
(60) Provisional application No. 60/948,999, filed on Jul. 11, 2007.

(51) Int. Cl. C12Q 1/68; C07H 21/04; C12N 15/63; C12N 5/10

(52) U.S. Cl. 435/6; 536/23.72; 435/320.1; 435/325

(57) ABSTRACT

The present invention discloses sequences having a structure and sequence homology to lox-P recombinase target site corresponding to a highly conserved region within the Long Terminal Repeats (LTR) of HIV and to the use thereof for the identification of recombinase enzymes useful in treating HIV-1.

### FIELD OF THE INVENTION

The present invention relates to an isolated polynucleotide having a sequence corresponding to a highly conserved region within the Long Terminal Repeat (LTR) of HIV and to the use thereof as a target sequence within HIV for its removal from the genome and for the identification of recombinase enzymes useful in treating Human Immunodeficiency Virus 1 (HIV-1).

### BACKGROUND OF THE INVENTION

Human immunodeficiency virus type 1 (HIV-1) replication is initiated through interaction of the viral envelope glycoprotein gp120 with its host cell receptors CD4 and CCR5. Fusion of viral and cellular membranes results in release of the nucleocapsid into the cytoplasm. Positive-stranded genomic RNA serves as template for synthesis of a cDNA by HIV-1 reverse transcriptase (RT). Following translocation of the cDNA into the nucleus, the viral integrase mediates insertion of the cDNA into cellular chromosomal DNA to produce the HIV-1 provirus. Establishment of the provirus is an obligatory step in HIV-1 replication and serves to maintain viral sequences within the infected cell after cellular division. The long terminal repeat (LTR) of proviral DNA directs the expression of viral genes, including those encoding components of the virion. During the process of viral integration the LTR is duplicated and flanks the viral genome on both sides. HIV-1 particles are assembled at the plasma membrane, followed by budding and proteolytic cleavage of viral structural proteins by the HIV-1 protease to produce mature virions.

The most conserved part of HIV-1 genome is found in the 5′ non-translated 335 base-pair (bp) leader located within the LTR regions. The leader RNA contains multiple sequence and structural motifs that play important roles in distinct steps of the viral replication cycle (FIG. 1). The leader region of all HIV mRNAs, whether spliced or unspliced, starts with the formation of a stem-loop structure called the trans-activating responsive (TAR) element that mediates transcriptional activation. The leader region also contains a poly(A) hairpin that suppresses a polyadenylation signal found therein, a major splice donor, and a start codon of the group-specific antigen (gag) open reading frame (ORF). Packaging of a dimeric form of the RNA genome is also controlled by sequences in the leader: the dimer initiation signal (DIS) hairpin and the psi signal. This region also contains a sequence known as the “primer binding site” (PBS) that controls reverse transcription by the HIV-encoded RT together with accessory sequence motifs such as the primer activation signal (PAS).

The human TAR RNA binding protein (TRBP), which is encoded by the human host genome, is an essential participant in Dicer and a crucial component of the RNA Induced Silencing Complex (RISC). Data obtained by examining the effect of TAR RNA on the RNAi machinery suggest that TAR RNA sequesters TRBP, rendering it unavailable for Dicer-RISC complexes and thus resistant to RNA interference (RNAi) (Bennasser et al., 2006).

Estable et al. (Estable et al. 1996. J. Virol. 70: 4053-4062) identified highly conserved motifs within the LTR: TATA box, SP-1 and the NF-kB sites. Mutations that alter the stability of the TAR stem region severely inhibit HIV-1 replication (Das et al. 1997. J Viro 171:2346-2356). The TAR stem-loop structure and conservation of sequences in the stem and the loop and the distance between the stem and the loop are all required for the trans-activation of TAR by the Tat transactivator protein (Bannwarth and Gatignol 2005. Curr HIV Res 3:61-71). Additionally, the poly(A) stem and loop structure and its thermodynamic stability are well conserved among HIV and SIV isolates despite considerable sequence divergence in the rest of the genome.

Anti-viral agents against HIV-1 have been targeted to virally encoded enzymes such as RT, integrase and protease. Compounds that inhibit RT or integrase may prevent the establishment of proviral DNA, while inhibitors of the viral protease block the maturation of virus particles. Chemical agents targeted to virally encoded enzymes may provide only transient inhibition of virus replication due to rapid generation of drug-resistant HIV-1 variants.

An alternative strategy has been use of antiviral genes delivered to uninfected cells as RNA or DNA, to provide intracellular protection from HIV-1 infection. Antiviral genes include those encoding antisense molecules, ribozymes, transdominant proteins and intracellular antibodies. It has been also suggested to introduce an expression vector encoding a recombinase that recognizes sequences within the LTR of proviral DNA. Due to the conserved nature of LTR elements and additionally, its repeated configuration flanking the viral genome at the provirus integration site, the recombinase could mediate excision of viral coding sequences, resulting in the elimination of the intact provirus from the infected cells (Flowers et al., 1997. J Virology 71(4):2685-2692).

The Cre-loxP recombination system of bacteriophage P1 catalyzes site-specific recombination in vitro, in eukaryotic cells and in transgenic animals. Only two components are required to mediate recombination: a recombinase—the Cre recombinase, and a sequence-specific recombinase target site—the loxP site. Cre can mediate either inter- or intramolecular recombination upon recognition of two loxP sites on either linear or supercoiled DNA. In the wild-type Cre-loxP system, the loxP site consists of two 13-pb inverted repeats flanking an asymmetric 8-bp spacer.

An endogenous lox-type sequence located within the HIV-1 LTR (loxP-like LTR sequence) was reported as a potential target for Cre recombinase excision reaction (Lee and Park 1998. Biochem. Biophys. Res. Commun. 253: 588-593; Lee et al., 2000. Cell Biol. 78: 653-658; Kim et al., 2001. J Cell Biochem. 80: 321-327). However, this reported target sequence is located in a non-conserved region of HIV-1 LTR. The non-conserved region is susceptible to mutations, and thus is not likely to be an effective target for anti-HIV-1 therapy.

Various mutated Cre enzymes and loxP sites, including that from Lee and Park 1998, have been described, as well as their activity and use in designing symmetric sites based on the half-sites found therein (Sarkar et al, 2007. HIV-1 proviral DNA excision using an evolved recombinase. Science. 316(5833):1912-5; Bucholtz and Stewart 2001. Nat. Biotechnol. 19:1047-1052; Hartung and Kisters-Woike 1998. J Biol. Chem. 273(36):22884-91). These references do not disclose or suggest in any way the highly conserved sequences of the present invention or their use in identifying Cre recombinases for broad-spectrum anti-HIV-1 activity.

International Patent Application WO 2005/081632 discloses enzymes, compositions and methods for catalyzing asymmetric recombination of non-palindromic recombination sites in a cell-free system, in isolated cells, or in living organisms. The enzymes and methods are suitable for mediating specific recombination between DNA sequences comprising specific recombination sites without being limited to strict palindromic symmetry within each recombination site. This reference does not disclose or suggest in any way the highly conserved sequences of the present invention or their use in identifying Cre recombinases for broad-spectrum anti-HIV-1 activity.

Thus, there is an unmet need for, and it would be highly advantageous to have, Cre-/oxP target sequences located within a conserved region of the HIV-1 genome.

### SUMMARY OF THE INVENTION

The present invention relates to a highly conserved nucleic acid sequence located within the long terminal repeat (LTR) region of human immunodeficiency virus 1 (HIV-1) and its use for developing means for the treatment of AIDS. Particularly, the present invention relates to a loxP-type sequence-specific recombinase target site and its use in selecting a compatible recombinase.

The present invention is based in part on the unexpected discovery of highly conserved region of about 50 by comprising a 34-bp lox-type sequence, located partially within the trans-activating responsive (TAR) stem and partially within the poly(A) stem of the HIV-1 LTR region. These sequences are conserved among HIV isolates and are therefore highly suitable as a target for therapeutic genetic manipulation. The structure and sequence resemblance to loxP, and their presence at the 5′-end as well as the 3′-end of the proviral DNA, enable the excision of a DNA segment by an active recombinase, thus inactivating the virus.

Similar to the lox-P structure, the structure of recombinase target sites of the present invention includes a first arm (Arm A) of 13 nucleotides, a spacer of 8 nucleotides and a second arm (Arm B) of 13 nucleotides. Each of these sequence segments is useful in the construction of new lox-P type polynucleotides, useful for identifying and isolating new recombinase enzymes.

In one embodiment, the present invention provides an isolated HIV-1 nucleic acid fragment comprising an HIV-1 nucleic acid sequence selected from the group consisting of TAACTAGGGAACC (SEQ ID NO:1), CACTGCTT (SEQ ID NO:2) and AAGCCTCAATAAA (SEQ ID NO:3), wherein the length of the isolated HIV-1 nucleic acid sequence is from about 30 to about 100 nucleic acids.

In another embodiment, the isolated HIV-1 nucleic acid fragment comprises the sequence TAACTAGGGAACCCACTGCTT (SEQ ID NO:28). In another embodiment, the isolated HIV-1 nucleic acid fragment comprises the sequence CACTGCTTAAGCCTCAATAAA (SEQ ID NO:29). In another embodiment, the isolated HIV-1 nucleic acid fragment comprises the sequence TAACTAGGGAACCCACTGCTTAAGCCTCAATAAA (SEQ ID NO:4). Each possibility represents a separate embodiment of the present invention. In another embodiment, the present invention provides a polynucleotide comprising the isolated HIV-1 nucleic acid fragment. Each possibility represents a separate embodiment of the present invention.

In another embodiment, the present invention provides a polynucleotide comprising the nucleic acid sequence TAACTAGGGAACCnnnnnnnGGTTCCCTAGTTA (SEQ ID NO:12). In another embodiment, the nucleic acid sequence is TAACTAGGGAACCGCATACATGGTTCCCTAGTTA (SEQ ID NO:5), containing the wt loxP spacer. In another embodiment, the nucleic acid sequence is TAACTAGGGAACCCACTGCTTGGTTCCCTAGTTA (SEQ ID NO:6), containing the spacer found in the wt LTR sequence. In another embodiment, the spacer nnnnnnnn is any other spacer compatible with Cre recombination. Each possibility represents a separate embodiment of the present invention.

The spacer of methods and compositions of the present invention is, in another embodiment, a chimera of spacers found in the loxP and LTR sequences. Non-limiting examples of chimeric spacers are GACTGCTT, GCCTGCTT, GCATGCTT, GCATGCTT, GCATACTT, and GCATACTT, derived from the spacers of SEQ ID NOs: 4 and 11; GACTGCTT, GCCTGCTT, GCCTGCTT, GCCTGCTT, GCCTCCTT, GCCTCATT, derived from the spacers of SEQ ID NOs: 9 and 11; AACTGCTT, AACTGCTT, AATTGCTT, AATAGCTT, AATAACTT, AATAAATT, AATAAAGT, derived from the spacers of SEQ ID NOs: 10 and 11. Each possibility represents a separate embodiment of the present invention.

In another embodiment, the present invention provides a polynucleotide comprising the nucleic acid sequence TTTATTGAGGCTTnnnnnnnnAAGCCTCAATAAA (SEQ ID NO:13). In another embodiment, the nucleic acid sequence is TTTATTGAGGCTTGCATACATAAGCCTCAATAAA (SEQ ID NO:7), containing the wt loxP spacer. In another embodiment, the nucleic acid sequence is TTTATTGAGGCTTCACTGCTTAAGCCTCAATAAA (SEQ ID NO:8), containing the spacer found in the wt LTR sequence. In another embodiment, the spacer nnnnnnnn is any other spacer compatible with Cre recombination. Each possibility represents a separate embodiment of the present invention.

In another embodiment, the present invention provides a polynucleotide comprising the nucleic acid sequence TAACTAGGGAACCnnnnnnnnAAGCCTCAATAAA (SEQ ID NO:31), wherein “nnnnnnnn” represents any combination of nucleic acid bases other than CACTGCTT. In another embodiment, the nucleic acid sequence is TAACTAGGGAACCGCATACATAAGCCTCAATAAA (SEQ ID NO:54), containing the wt loxP spacer. In another embodiment, the spacer is any spacer compatible with Cre recombination other than CACTGCTT. Each possibility represents a separate embodiment of the present invention.

According to one embodiment, a polynucleotide of the present invention comprises a nucleic acid sequence as set forth in SEQ ID NO:1 (Arm A). According to certain embodiments, the isolated polynucleotide has a nucleic acid sequence selected from the group consisting of TAACTAGGGAACCCACTGCTTAAGCCTCAATAAA (SEQ ID NO: 4); TAACTAGGGAACCGCATACATGGTTCCCTAGTTA (SEQ ID NO:5) TAACTAGGGAACCCACTGCTTGGTTCCCTAGTTA (SEQ ID NO:6), and TAACTAGGGAACCnnnnnnnnGGTTCCCTAGTTA (SEQ ID NO:12).

According to another embodiment, the isolated polynucleotide comprises SEQ ID NO: 3 (Arm B). According to certain embodiments, the isolated polynucleotide has a nucleic acid sequence selected from the group consisting of TAACTAGGGAACCCACTGCTTAAGCCTCAATAAA (SEQ ID NO: 4); TTTATTGAGGCTTGCATACATAAGCCTCAATAAA (SEQ ID NO:7); TTTATTGAGGCTTCACTGCTTAAGCCTCAATAAA (SEQ ID NO:8); and TTTATTGAGGCTTnnnnnnnnAAGCCTCAATAAA (SEQ ID NO:13).

According to certain embodiments, the isolated polynucleotide is of the sequence TAACTAGGGAACCCACTGCTTAAGCCTCAATAAA (SEQ ID NO: 4).

The highly conserved region of the HIV-1 LTR region comprises further sequences having a structural homology to the lox-P recombination site.

In another embodiment, the present invention provides an isolated HIV-1 nucleic acid fragment comprising the HIV-1 nucleic acid sequence ACCCACTGCTTAAGCCTCAATAAAGCTTGCCTTG (SEQ ID NO:9), wherein the length of the isolated HIV-1 nucleic acid sequence is from about 30 to about 100 nucleic acids. In another embodiment, the present invention provides a polynucleotide comprising the isolated HIV-1 nucleic acid fragment. Each possibility represents a separate embodiment of the present invention.

SEQ ID NO:9 has a first arm of nucleotides 1-13 (Arm A) (ACCCACTGCTTAA; SEQ ID NO:18), a spacer of nucleotides 14-21 (GCCTCAAT; SEQ ID NO:19) and a second arm of nucleotides 22-34 (Arm B) (AAAGCTTGCCTTG; SEQ ID NO:20).

In another embodiment, the present invention provides a polynucleotide comprising the nucleic acid sequence ACCCACTGCTTAAnnnnnnnnAAAGCTTGCCTTG (SEQ ID NO:14), wherein “nnnnnnnn” represents any combination of nucleic acid bases other than GCCTCAAT. In another embodiment, the nucleic acid sequence is ACCCACTGCTTAAGCATACATAAAGCTTGCCTTG (SEQ ID NO:52), containing the wt loxP spacer. In another embodiment, the spacer is any spacer compatible with Cre recombination other than GCCTCAAT. Each possibility represents a separate embodiment of the present invention.

In another embodiment, the present invention provides an isolated HIV-1 nucleic acid fragment comprising the HIV-1 nucleic acid sequence CTGCTTAAGCCTCAATAAAGCTTGCCTTGAGTGC (SEQ ID NO:10), wherein the length of the isolated HIV-1 nucleic acid sequence is from about 30 to about 100 nucleic acids. In another embodiment, the present invention provides a polynucleotide comprising the isolated HIV-1 nucleic acid fragment. Each possibility represents a separate embodiment of the present invention.

SEQ ID NO:10 has a first arm of nucleotides 1-13 (Arm A) (CTGCTTAAGCCTC; SEQ ID NO:21), a spacer of nucleotides 14-21 (AATAAAGC; SEQ ID NO:22) and a second arm of nucleotides 22-34 (Arm B) (TTGCCTTGAGTGC; SEQ ID NO:23).

In another embodiment, the present invention provides a polynucleotide comprising the nucleic acid sequence CTGCTTAAGCCTCnnnnnnnnTTGCCTTGAGTGC (SEQ ID NO:15), wherein “nnnnnnnn” represents any combination of nucleic acid bases other than AATAAAGC. In another embodiment, the nucleic acid sequence is CTGCTTAAGCCTCGCATACATTTGCCTTGAGTGC (SEQ ID NO:53), containing the wt loxP spacer. In another embodiment, the spacer is any spacer compatible with Cre recombination other than AATAAAGC. Each possibility represents a separate embodiment of the present invention.

In another embodiment, the present invention provides a polynucleotide comprising the nucleic acid sequence ACCCACTGCTTAAnnnnnnnnTTAAGCAGTGGGT (SEQ ID NO:40). In another embodiment, the nucleic acid sequence is ACCCACTGCTTAAGCCTCAATTTAAGCAGTGGGT (SEQ ID NO:41), containing the spacer found in the wt LTR sequence. In another embodiment, the nucleic acid sequence is ACCCACTGCTTAAGCATACATTTAAGCAGTGGGT (SEQ ID NO:42), containing the wt loxP spacer. In another embodiment, the spacer is any other spacer compatible with Cre recombination. Each possibility represents a separate embodiment of the present invention.

In another embodiment, the present invention provides a polynucleotide comprising the nucleic acid sequence CAAGGCAAGCTTTnnnnnnnnAAAGCTTGCCTTG (SEQ ID NO:43). In another embodiment, the nucleic acid sequence is CAAGGCAAGCTTTGCCTCAATAAAGCTTGCCTTG (SEQ ID NO:44), containing the spacer found in the wt LTR sequence. In another embodiment, the nucleic acid sequence is CAAGGCAAGCTTTGCATACATAAAGCTTGCCTTG (SEQ ID NO:45), containing the wt loxP spacer. In another embodiment, the spacer is any other spacer compatible with Cre recombination. Each possibility represents a separate embodiment of the present invention.

In another embodiment, the present invention provides a polynucleotide comprising the nucleic acid sequence CTGCTTAAGCCTCnnnnnnnnGAGGCTTAAGCAG (SEQ ID NO:46). In another embodiment, the nucleic acid sequence is CTGCTTAAGCCTCAATAAAGCGAGGCTTAAGCAG (SEQ ID NO:47), containing the spacer found in the wt LTR sequence. In another embodiment, the nucleic acid sequence is CTGCTTAAGCCTCGCATACATGAGGCTTAAGCAG (SEQ ID NO:48), containing the wt loxP spacer. In another embodiment, the spacer is any other spacer compatible with Cre recombination. Each possibility represents a separate embodiment of the present invention.

In another embodiment, the present invention provides a polynucleotide comprising the nucleic acid sequence GCACTCAAGGCAAnnnnnnnnTTGCCTTGAGTGC (SEQ ID NO:49). In another embodiment, the nucleic acid sequence is GCACTCAAGGCAAAATAAAGCTTGCCTTGAGTGC (SEQ ID NO:50), containing the spacer found in the wt LTR sequence.). In another embodiment, the nucleic acid sequence is GCACTCAAGGCAAGCATACATTTGCCTTGAGTGC (SEQ ID NO:51), containing the wt loxP spacer. In another embodiment, the spacer is any other spacer compatible with Cre recombination. Each possibility represents a separate embodiment of the present invention.

In another embodiment, the present invention provides a nucleic acid construct comprising a sequence as set forth in any one of SEQ ID NO:4, SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, or a sequence complementary thereto. According to one embodiment, the construct comprises a double stranded polynucleotide.

As provided herein, sequences of the present invention are useful for the detection and isolation of a recombinase capable of catalyzing a recombination between two sequence-specific recombinase target sites.

According to other embodiments, the nucleic acid construct is an expression vector. According to additional embodiments, there is provided a host cell comprising the nucleic acid construct of the invention.

An excision of a DNA fragment flanked by a pair of the lox-P type recombination sequence of the present invention requires selection of a recombinase that recognizes this sequence and catalyzes recombination.

The method of the present invention teaches the use of a sequence-specific recombinase target site for identifying a recombinase capable of excising HIV-1 viral coding sequences. Any method as is known in the art for identifying excision of a certain DNA segment from a specific polynucleotide can be used with systems or methods provided by the present invention. According to certain embodiment, excision is detected using a PCR reaction with pre-designed primers. According to other embodiments, excision is detected by digesting the isolated nucleic acid construct with restriction enzymes and analyzing the digestion products. Analyzing the digestion products may be performed using, for example, PCR reaction, size fractionation, etc., and any combination thereof.

Other objects, features and advantages of the present invention will become clear from the following description and drawings.

### BRIEF DESCRIPTION OF THE FIGURES

FIG. 1 shows a schematic representation of the HIV-1 5′ non-translated leader RNA (Beerens et al. (2001) J Biol. Chem. 276: 31247-31256).

FIG. 2 is a schematic illustration of pBAD33 vector cloned with wt Cre and two identical 34 bp inverted repeats of lox-LTR flanking an intervening DNA fragment of 260 bp. Primer binding sites for PCR, allowing differentiation of recombined from non-recombined DNA substrate, are marked by arrows.

FIG. 3 shows the various chimeric mutated loxP-like LTR sequences (B) compared to the wild type lox-P (SEQ ID NO:11) and to loxP-like LTR (SEQ ID NO:4) sequences (A). Sequences in (B) are SEQ ID NOS: 16-17 and 32-39, respectively

FIG. 4 A-B shows restriction map and PCR analysis, presenting recombination products of wt Cre in LTR4 constructs. loxP and 10 loxP-like LTR substrates were cloned into pBAD33. 1—loxP; 2-LTRspacer; 3-LTRa 1-5; 4—LTRa 1-6; 5—LTRa 9-13; 6—LTR4a 6-8; 7—LTRa 7-13; 8—LTR4b 1-5; 9—LTRb1-6; 10—LTRb 7-13; 11—LTRb 6-8. Each chimeric substrate was subjected to recombination assay with wt Cre as: uncleaved plasmid (a), digestion with NdeI (b) digestion with NdeI+NcoI (c). (A) Restriction map analysis of the 11 substrates. (B) PCR analysis of the 11 substrates.

FIG. 5. Validation of in vitro Cre recombinase assay. Red arrows mark fragments obtained following recombination activity. Upper band (˜4 kb) is the full substrate; fragment below it (˜3 kb) is the substrate plasmid without the excised ˜1 kb fragment. Fragments at the bottom are excised fragment itself. ssDNA˜salmon sperm DNA; demo˜control plasmid that does not contain Cre recombinase; MW˜size markers.

FIG. 6. In vitro testing of activity of Cre variants. The ˜1200 bp fragment is from the non-recombined substrate, while the ˜200 bp fragment is from the recombined plasmid.

FIG. 7. Results from a lower-background in vitro assay. Arrows mark ˜250 bp fragment indicative of the recombination activity. The fragment of the non-recombined plasmid substrate cannot be seen, since the size of ˜3200 bp is not amplified in the PCR reaction used. BX is the plasmid that contained no Cre gene and was employed as negative control. PCR control is a reaction containing all PCR ingredients without addition of any recombination reaction.

FIG. 8. Homology of Glade representative sequences the region containing loxP-like LTR sequences of the present invention and that from Sarkar et al.

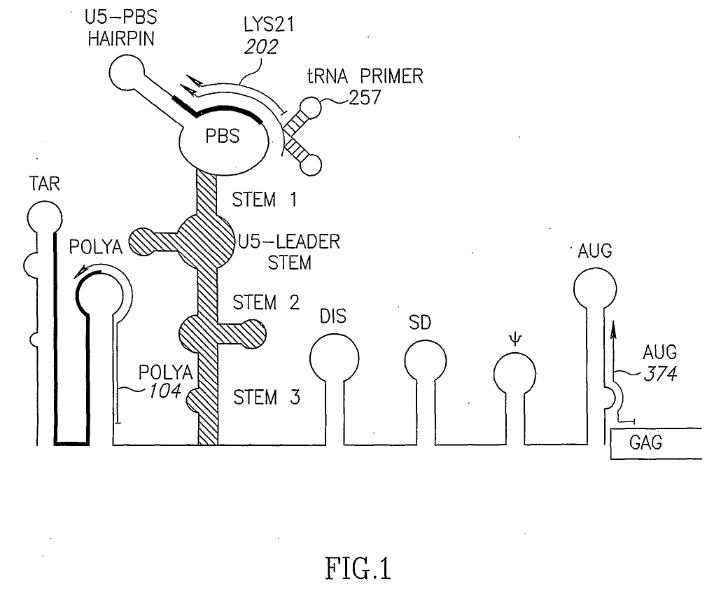

### DETAILED DESCRIPTION OF THE INVENTION

The present invention discloses a nucleic acid sequence located within a highly conserved region of HIV-1 LTR, having sequence and structure homology to the loxP site specific recombination sequence, and thus serving as a target to corresponding Cre-analog enzymes. The present invention further provides means and methods for the identification and isolation of recombinase enzymes capable of excising a DNA segment flanked by this LoxP-type site-specific recombination sequences.

In one embodiment, the present invention provides an isolated HIV-1 nucleic acid fragment comprising an HIV-1 nucleic acid sequence selected from the group consisting of TAACTAGGGAACC (SEQ ID NO:1), CACTGCTT (SEQ ID NO:2) and AAGCCTCAATAAA (SEQ ID NO:3), wherein the length of the isolated HIV-1 nucleic acid sequence is from about 30 to about 100 nucleic acids.

In another embodiment, the isolated HIV-1 nucleic acid fragment comprises the sequence TAACTAGGGAACCCACTGCTT (SEQ ID NO:28). In another embodiment, the isolated HIV-1 nucleic acid fragment comprises the sequence CACTGCTTAAGCCTCAATAAA (SEQ ID NO:29). In another embodiment, the isolated HIV-1 nucleic acid fragment comprises the sequence TAACTAGGGAACCCACTGCTTAAGCCTCAATAAA (SEQ ID NO:4). Each possibility represents a separate embodiment of the present invention. In another embodiment, the present invention provides a polynucleotide comprising the isolated HIV-1 nucleic acid fragment. Each possibility represents a separate embodiment of the present invention.

In another embodiment, the present invention provides a polynucleotide comprising the nucleic acid sequence TAACTAGGGAACCnnnnnnnnAAGCCTCAATAAA (SEQ ID NO:31), wherein “nnnnnnnn” represents any combination of nucleic acid bases other than CACTGCTT. In another embodiment, the nucleic acid sequence is TAACTAGGGAACCGCATACATAAGCCTCAATAAA (SEQ ID NO:54), containing the wt loxP spacer. In another embodiment, the spacer is any spacer compatible with Cre recombination other than CACTGCTT. Each possibility represents a separate embodiment of the present invention.

In another embodiment, the present invention provides a polynucleotide comprising the nucleic acid sequence TAACTAGGGAACCnnnnnnnnGGTTCCCTAGTTA (SEQ ID NO:12). In another embodiment, the nucleic acid sequence is TAACTAGGGAACCGCATACATGGTTCCCTAGTTA (SEQ ID NO:5), containing the wt loxP spacer. In another embodiment, the nucleic acid sequence is TAACTAGGGAACCCACTGCTTGGTTCCCTAGTTA (SEQ ID NO:6), containing the spacer found in the wt LTR sequence. In another embodiment, the spacer is any other spacer compatible with Cre recombination. Each possibility represents a separate embodiment of the present invention.

The spacer of methods and compositions of the present invention is, in another embodiment, a spacer found in a loxP-like LTR sequence of the present invention. In another embodiment, the spacer is selected from the group consisting of CACTGCTT (SEQ ID NO:2), GCCTCAAT, and AATAAAGC.

In another embodiment, the spacer is a chimera of the loxP and loxP-like LTR sequences. Non-limiting examples of chimeric spacers are GACTGCTT, GCCTGCTT, GCATGCTT, GCATGCTT, GCATACTT, and GCATACTT, derived from the spacers of SEQ ID NOs: 4 and 11; GACTGCTT, GCCTGCTT, GCCTGCTT, GCCTGCTT, GCCTCCTT, GCCTCATT, derived from the spacers of SEQ ID NOs: 9 and 11; AACTGCTT, AACTGCTT, AATTGCTT, AATAGCTT, AATAACTT, AATAAATT, AATAAAGT, derived from the spacers of SEQ ID NOs: 10 and 11. In another embodiment, the spacer is selected from the group consisting of GACTGCTT, GCCTGCTT, GCATGCTT, GCATGCTT, GCATACTT, GCATACTT, GACTGCTT, GCCTGCTT, GCCTGCTT, GCCTGCTT, GCCTCCTT, GCCTCATT, AACTGCTT, AACTGCTT, AATTGCTT, AATAGCTT, AATAACTT, AATAAATT, and AATAAAGT. Each possibility represents a separate embodiment of the present invention.

In another embodiment, the present invention provides a polynucleotide comprising the nucleic acid sequence TTTATTGAGGCTTnnnnnnnnAAGCCTCAATAAA (SEQ ID NO:13). In another embodiment, the nucleic acid sequence is TTTATTGAGGCTTGCATACATAAGCCTCAATAAA (SEQ ID NO:7), containing the wt loxP spacer. In another embodiment, the nucleic acid sequence is TTTATTGAGGCTTCACTGCTTAAGCCTCAATAAA (SEQ ID NO:8), containing the spacer found in the wt LTR sequence. In another embodiment, the spacer is any other spacer compatible with Cre recombination. Each possibility represents a separate embodiment of the present invention.

In another embodiment, the present invention provides a polynucleotide comprising the nucleic acid sequence ACCCACTGCTTAAnnnnnnnnAAAGCTTGCCTTG (SEQ ID NO:14), wherein “nnnnnnnn” represents any combination of nucleic acid bases other than GCCTCAAT. In another embodiment, the nucleic acid sequence is ACCCACTGCTTAAGCATACATAAAGCTTGCCTTG (SEQ ID NO:52), containing the wt loxP spacer. In another embodiment, the spacer is any spacer compatible with Cre recombination other than GCCTCAAT. Each possibility represents a separate embodiment of the present invention.

In another embodiment, the present invention provides an isolated HIV-1 nucleic acid fragment comprising the HIV-1 nucleic acid sequence ACCCACTGCTTAAGCCTCAATAAAGCTTGCCTTG (SEQ ID NO:9), wherein the length of the isolated HIV-1 nucleic acid sequence is from about 30 to about 100 nucleic acids. In another embodiment, the present invention provides a polynucleotide comprising the isolated HIV-1 nucleic acid fragment. Each possibility represents a separate embodiment of the present invention.

In another embodiment, the present invention provides a polynucleotide comprising the nucleic acid sequence CTGCTTAAGCCTCnnnnnnnnTTGCCTTGAGTGC (SEQ ID NO:15), wherein “nnnnnnnn” represents any combination of nucleic acid bases other than AATAAAGC. In another embodiment, the nucleic acid sequence is CTGCTTAAGCCTCGCATACATTTGCCTTGAGTGC (SEQ ID NO:53), containing the wt loxP spacer. In another embodiment, the spacer is any spacer compatible with Cre recombination other than AATAAAGC. Each possibility represents a separate embodiment of the present invention.

In another embodiment, the present invention provides an isolated HIV-1 nucleic acid fragment comprising the HIV-1 nucleic acid sequence CTGCTTAAGCCTCAATAAAGCTTGCCTTGAGTGC (SEQ ID NO:10), wherein the length of the isolated HIV-1 nucleic acid sequence is from about 30 to about 100 nucleic acids. In another embodiment, the present invention provides a polynucleotide comprising the isolated HIV-1 nucleic acid fragment. Each possibility represents a separate embodiment of the present invention.

In another embodiment, the present invention provides a polynucleotide comprising the nucleic acid sequence ACCCACTGCTTAAnnnnnnnnTTAAGCAGTGGGT (SEQ ID NO:40). In another embodiment, the nucleic acid sequence is ACCCACTGCTTAAGCCTCAATTTAAGCAGTGGGT (SEQ ID NO:41), containing the spacer found in the wt LTR sequence. In another embodiment, the nucleic acid sequence is ACCCACTGCTTAAGCATACATTTAAGCAGTGGGT (SEQ ID NO:42), containing the wt loxP spacer. In another embodiment, the spacer is any other spacer compatible with Cre recombination. Each possibility represents a separate embodiment of the present invention.

In another embodiment, the present invention provides a polynucleotide comprising the nucleic acid sequence CAAGGCAAGCTTTnnnnnnnnAAAGCTTGCCTTG (SEQ ID NO:43). In another embodiment, the nucleic acid sequence is CAAGGCAAGCTTTGCCTCAATAAAGCTTGCCTTG (SEQ ID NO:44), containing the spacer found in the wt LTR sequence. In another embodiment, the nucleic acid sequence is CAAGGCAAGCTTTGCATACATAAAGCTTGCCTTG (SEQ ID NO:45), containing the wt loxP spacer. In another embodiment, the spacer is any other spacer compatible with Cre recombination. Each possibility represents a separate embodiment of the present invention.

In another embodiment, the present invention provides a polynucleotide comprising the nucleic acid sequence CTGCTTAAGCCTCnnnnnnnnGAGGCTTAAGCAG (SEQ ID NO:46). In another embodiment, the nucleic acid sequence is CTGCTTAAGCCTCAATAAAGCGAGGCTTAAGCAG (SEQ ID NO:47), containing the spacer found in the wt LTR sequence. In another embodiment, the nucleic acid sequence is CTGCTTAAGCCTCGCATACATGAGGCTTAAGCAG (SEQ ID NO:48), containing the wt loxP spacer. In another embodiment, the spacer is any other spacer compatible with Cre recombination. Each possibility represents a separate embodiment of the present invention.

In another embodiment, the present invention provides a polynucleotide comprising the nucleic acid sequence GCACTCAAGGCAAnnnnnnnnTTGCCTTGAGTGC (SEQ ID NO:49). In another embodiment, the nucleic acid sequence is GCACTCAAGGCAAAATAAAGCTTGCCTTGAGTGC (SEQ ID NO:50), containing the spacer found in the wt LTR sequence.). In another embodiment, the nucleic acid sequence is GCACTCAAGGCAAGCATACATTTGCCTTGAGTGC (SEQ ID NO:51), containing the wt loxP spacer. In another embodiment, the spacer is any other spacer compatible with Cre recombination. Each possibility represents a separate embodiment of the present invention.

In another embodiment, the present invention provides a polynucleotide comprising an isolated HIV-1 nucleic acid fragment of the present invention.

In another embodiment, the present invention provides an expression vector comprising an isolated HIV-1 nucleic acid fragment of the present invention.

In another embodiment, the present invention provides a host cell comprising an isolated HIV-1 nucleic acid fragment of the present invention.

In another embodiment, the present invention provides an expression vector comprising a polynucleotide of the present invention.

In another embodiment, the present invention provides a host cell comprising a polynucleotide of the present invention. Any host cells suitable for harboring and expressing polynucleotides as are known in the art can be used according to the teaching of the present invention. According to certain embodiments, the host cells are selected from eukaryotic cells and prokaryotic cells. According to certain currently preferred embodiments, the cells are bacterial cells. Each possibility represents a separate embodiment of the present invention.

In another embodiment, the present invention provides a kit for measuring the recombinase activity of an enzyme, the kit comprising a plurality of host cells, each cell comprising a polynucleotide containing a target sequence of the present invention. In another embodiment, the polynucleotide comprises an isolated HIV-1 nucleic acid fragment. In another embodiment, the polynucleotide comprises SEQ ID NO:12. In another embodiment, the polynucleotide comprises SEQ ID NO:13. In another embodiment, the polynucleotide comprises SEQ ID NO:14. In another embodiment, the polynucleotide comprises SEQ ID NO:15. In another embodiment, the polynucleotide comprises any other nucleic acid sequence of the present invention. In another embodiment, the recombinase enzyme is a Cre recombinase. In another embodiment, the kit further comprises instructions for measuring the recombinase activity of an enzyme Each possibility represents a separate embodiment of the present invention.

In another embodiment, the host cells in the kit comprise an expression vector of the present invention. In another embodiment, the expression vector is the same construct as the polynucleotide that contains the target sequence. In another embodiment, the expression vector and the polynucleotide that contains the target sequence are on separate constructs. In another embodiment, the expression vectors comprise a transcribable polynucleotide sequence encoding one recombinase of a plurality of recombinases.

According to yet other embodiments, the method of the present invention employs a combined nucleic acid construct comprising an additional polynucleotide sequence encoding a polypeptide capable of conferring resistance to an antibiotic, wherein the polynucleotides is operably linked to its promoter only after excision has been occurred as described hereinabove. In these embodiments, excision is detected by growing the host cell comprising said combined nucleic acid construct in an antibiotic-comprising medium.

According to certain currently preferred embodiments, the combined nucleic acid construct comprises:

a) a first nucleic acid construct comprising a transcribable polynucleotide sequence encoding one recombinase of a plurality of recombinases, the first transcribable polynucleotide sequence being operatively linked to a first promoter; and

b) a second nucleic acid construct comprising a second promoter; a DNA segment having a 5′-end and a 3′-end wherein the DNA segment is flanked by a pair of sequence-specific recombinase target sites, one at the 5′-end and one at the 3′-end, and wherein the recombinase target site has a sequence as set forth in SEQ ID NO:4; and a second transcribable polynucleotide sequence encoding a polypeptide capable of conferring resistance to an antibiotic;

wherein the second promoter and the second transcribable polynucleotides are operably linked only after excision of said DNA segment by the recombinase.

### DEFINITIONS

The term “recombinase” as used herein is to be construed in its most general sense, and refers to an enzyme or a plurality of enzymes, active fragments or active variants thereof, capable of catalyzing recombination events between two sequence-specific recombinase target sites. In fact, a recombinase is an enzyme capable of catalyzing cleavage and ligation at particular sites.

As used herein the terms “recombinase” “sequence-specific recombinase” and “site-specific recombinase” refer to enzymes that recognize and bind to a specific recombination site or sequence and catalyze the recombination of nucleic acid in relation to these sites.

The terms “sequence-specific recombinase target site” and “site-specific recombinase target site” and “recombinase target site” refer to short nucleic acid site or sequence which is recognized by a sequence- or site-specific recombinase and which become the crossover regions during the site-specific recombination event. Examples of sequence-specific recombinase target sites include, but are not limited to, lox sites, frt sites, ATT sites and DIF sites. According to certain embodiments, the target sites comprises a nucleic acid sequence selected from the group consisting of SEQ ID NO:4, SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51.

As used herein the terms “loxP” and “loxP site” are used interchangeably to describe a nucleotide sequence at which the product of the cre gene of bacteriophage P1, Cre recombinase or mutants thereof, can catalyze a site-specific recombination. The loxP site comprises two 13 by inverted repeat sequences separated by an 8 by spacer region (Hoess et al., Proc. Natl. Acad. Sci. USA 79:3398, 1982). The internal spacer sequence of the loxP site is asymmetrical and thus, two loxP sites can exhibit directionality relative to one another (Hoess et al. Proc. Natl. Acad. Sci. USA 81:1026, 1984). When two loxP sites on the same DNA molecule are in a directly repeated orientation, Cre excises the DNA between these two sites leaving a single loxP site on the DNA molecule. (Abremski et al. Cell 32:1301, 1983). If two loxP sites are in opposite orientation on a single DNA molecule, Cre inverts the DNA sequence between these two sites rather than removing the sequence. The terms “lox” or “lox site” encompass a variety of mutant lox sites as are known in the art including loxB, loxL and loxR (these are found in the E. coli chromosome) as well as a number of mutant or variant lox sites such as loxP511, loxΔ86, loxΔ117, loxC2, loxP2, loxP3 and loxP23. The Cre recombinase also recognizes a number of variant or mutant lox sites relative to the loxP sequence. Examples of these Cre recombination sites include, but are not limited to, the loxB, loxL and loxR sites which are found in the E. coli chromosome. The term “lox-type sequence-specific recombinase target site” refers to a sequence having the structure of loxP, i.e. having the structure of two arms, each having a sequence of 13 by separated by an 8 by spacer region, sharing at least 32% homology with the wild type loxP sequence.

The term “nucleic acid” as used herein refers to RNA or DNA that is linear or branched, single or double stranded, or a hybrid thereof. The term also encompasses RNA/DNA hybrids.

An “isolated” nucleic acid molecule is one that is substantially separated from other nucleic acid molecules which are present in the natural source of the nucleic acid (i.e., sequences encoding other polypeptides). Preferably, an “isolated” nucleic acid is free of some of the sequences that naturally flank the nucleic acid (i.e., sequences located at the 5′ and 3′ ends of the nucleic acid) in its naturally occurring replicon. For example, a cloned nucleic acid is considered isolated. A nucleic acid is also considered isolated if it has been altered by human intervention, or placed in a locus or location that is not its natural site, or if it is introduced into a cell by transformation or transfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

“HIV-1 nucleic acid sequence” refers to a nucleic acid sequence isolated from an HIV-1 virus. The term will be understood to encompass variants of naturally occurring HIV-1 sequences that retain substantial homology to the naturally occurring sequence. Preferably, greater than 70% homology is retained. In another embodiment, greater than 75% homology is retained. In another embodiment, greater than 80% homology is retained. In another embodiment, greater than 85% homology is retained. In another embodiment, greater than 90% homology is retained. In another embodiment, greater than 95% homology is retained. In another embodiment, greater than 98% homology is retained. Each possibility represents a separate embodiment of the present invention.

As used herein, the term “nucleic acid construct” refers to a nucleic acid molecules comprising several distinct segments and/or elements. The term includes circular nucleic acid constructs such as plasmid constructs, viral vector constructs, cosmid vectors, etc. as well as linear nucleic acid constructs (e.g., λ-phage constructs, PCR products). According to certain embodiment, the nucleic acid construct of the present invention is an expression vector that includes sequences that render this vector suitable for replication and integration in prokaryotes. In another embodiment, the expression vector is suitable for expression in eukaryotes. In another embodiment, the expression vector is suitable for expression in both prokaryotes and eukaryotes (e.g., a shuttle vector). The expression vector also contains expression signals such as a promoter and/or an enhancer. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “transforming” refers to DNA transfer to a host, achieved by any method known in the art, including but not limited to, transfection of DNA by calcium phosphate-precipitates, conventional mechanical procedures such as microinjection, electroporation or lipofection, insertion of a plasmid encapsulated in liposomes and the use of virus vectors for infection.

The term “host cell” refers to cells capable of growth in culture and capable of expressing an enzyme or a plurality of enzymes capable of mediating site-specific recombination between two predetermined recombination sites, wherein at least one of the recombination sites is an asymmetric recombination site. The host cells of the present invention include prokaryotic, eukaryotic, and mammalian cells. A host cell strain may be chosen which modulates the expression of the inserted sequences, or modifies and processes the gene product in the specific fashion desired. Expression from certain promoters can be elevated in the presence of certain inducers (e.g., zinc and cadmium ions for metallothionine promoters). Therefore expression of the enzyme or plurality of enzymes of the invention may be controlled. Different host cells have characteristic and specific mechanisms for the post-translational processing and modification of protein. Appropriate cell lines or host systems can be chosen to ensure the correct processing of enzymes expressed.

### Preferred Modes of Carrying Out the Invention

Hitherto known strategies to inhibit replication of HIV target either viral RNAs or proteins but do not affect the integrated proviral DNA. Employing these strategies requires the production of large quantities of the antiviral product in a sustained mode to continually inhibit viral replication.

The present invention provides recombinase target sequences within a highly conserved region of the HIV-1 LTR region that are useful for identifying recombinases capable of recognizing and binding to this sequence and catalyzing the recombination of nucleic acid in relation to at least two such sites. A recombinase such identified is useful in therapeutic for removing the proviral HIV-1 genome from the host genome. The HIV-1 genome removed from the host genome is expected to be degraded and disappear from the host cells.

The sequence-specific recombinase target site disclosed for the first time by present invention is located within a highly conserved region of about 50 bp, located partially within the TAR stem and partially within the poly(A) stem of the long terminal repeat (LTR) of HIV-1. Resembling the structure of lox-P, the recombinase target sites of the present invention comprise two arms of 13 by each, designated herein Arm A and Arm B, separated by a spacer of 8 bp. Polynucleotides having various combinations of these sequences with or without additional nucleic acid sequences are useful for the identification of recombinase enzymes capable of recognizing these target sites. The presence of LTR at the 5′-end and the 3′-end of the proviral DNA, and thus the presence of two recombinase target sites enables a recombination event to occur in the presence of a suitable recombinase, which recognizes the target site.

According to one aspect, the present invention provides an isolated polynucleotide of a sequence selected from the group consisting of TAACTAGGGAACC (SEQ ID NO:1, Arm A), CACTGCTT (SEQ ID NO:2, spacer) and AAGCCTCAATAAA (SEQ ID NO:3, Arm B) or a sequence complementary thereto.

According to one embodiment, the present invention provides an isolated polynucleotide of from 34 to about 100 nucleic acid comprising the nucleic acid sequence TAACTAGGGAACCnnnnnnnnAAGCCTCAATAAA (SEQ ID NO:31). In another embodiment, the isolated HIV-1 nucleic acid fragment comprises the sequence TAACTAGGGAACCCACTGCTTAAGCCTCAATAAA (SEQ ID NO:4) or a sequence complementary thereto and fragments thereof. SEQ ID NO:4 includes 13 nucleotides of Arm A (TAACTAGGGAACC; SEQ ID NO:1), 8 nucleotides being a spacer (CACTGCTT; SEQ ID NO:2) found in the HIV-1 sequence, and 13 nucleotides being Arm B (AAGCCTCAATAAA; SEQ ID NO:3). In another embodiment, any other spacer compatible with Cre recombinase activity if present instead of SEQ ID NO: 2. Each possibility represents a separate embodiment of the present invention.

According to one embodiment, the present invention provides an isolated polynucleotide of from 34 to about 100 nucleic acid comprising SEQ ID NO:1 (Arm A). According to certain embodiments, the isolated polynucleotide has a nucleic acid sequence selected from the group consisting of TAACTAGGGAACCCACTGCTTAAGCCTCAATAAA (SEQ ID NO: 4); TAACTAGGGAACCGCATACATGGTTCCCTAGTTA (SEQ ID NO:5) and TAACTAGGGAACCCACTGCTTGGTTCCCTAGTTA (SEQ ID NO:6).

The polynucleotide having SEQ ID NO:5 comprises a first segment of 13 by having the nucleic acid sequence set forth in SEQ ID NO: 1 (Arm A), a second segment of 8 by being the spacer of wild type LoxP (5′ ATAACTTCGTATAGCATACATTATACGAAGTTAT 3; SEQ ID NO:11), and a third segment of 13 by being an inverted repeat of SEQ ID NO:1 (Arm A).

The polynucleotide having SEQ ID NO:6 comprises a first segment of 13 by having the nucleic acid sequence set forth in SEQ ID NO: 1 (Arm A), a second segment of 8 by being a spacer of a nucleotide sequence as set forth in SEQ ID NO:2, and a third segment of 13 by being an inverted repeat of SEQ ID NO:1 (Arm A).

The polynucleotide having SEQ ID NO:7 comprises a first segment of 13 by being an inverted repeat of the nucleic acid sequence set forth in SEQ ID NO:3 (Arm B), a second segment of 8 by being the spacer of wild type LoxP (SEQ ID NO:11), and a third segment of 13 by having the nucleic acid sequence of Arm B (SEQ ID NO:3).

The polynucleotide having SEQ ID NO:8 comprises a first segment of 13 by having the nucleic acid sequence set forth in SEQ ID NO:3 (Arm B), a second segment of 8 by being a spacer of a nucleic acid sequence as set forth in SEQ ID NO:2, and a third segment of 13 by being an inverted repeat of SEQ ID NO:3 (Arm B).

According to yet further aspect, the present invention provides an isolated polynucleotide of from 34 to about 100 nucleic acid comprising the nucleic acid sequence ACCCACTGCTTAAGCCTCAATAAAGCTTGCCTTG (SEQ ID NO:9).

According to another aspect, the present invention provides an isolated polynucleotide of from 34 to about 100 nucleic acid comprising the nucleic acid sequence CTGCTTAAGCCTCAATAAAGCTTGCCTTGAGTGC (SEQ ID NO:10).

The sequence comprising the recombinase target sites of the present invention is highly conserved in various HIV clades as is evident from a search of this sequence within HIV-available sequence (Table 1).

TABLE 1
Homology to the LTR region of LTR4 of various HIV viruses using BLAST.

| Accession | Description | Max score | Total score |
|---|---|---|---|
| EF363124.1 | HIV-1 clone ES4-24 from USA, complete genome | 67.9 | 135 |
| EF363123.1 | HIV-1 clone ES1-20 from USA, complete genome | 67.9 | 135 |
| EF363122.1 | HIV-1 clone ES1-16 from USA, complete genome | 67.9 | 135 |
| DQ354122.1 | HIV-1 isolate MU2003 subtype CRF01_AE/B | 67.9 | 67.9 |
| | recombinant from Thailand gag protein (gag) and | | |
| | pol protein (pol) genes, partial cds; and vif protein | | |
| | (vif), vpr protein (vpr), tat protein (tat), rev protein | | |
| | (rev), vpu protein (vpu), envelope glycoprotein (env), | | |
| | and nef protein (nef) genes, complete cds | | |
| DQ336092.1 | HIV-1 isolate Y9 from France LTR, partial sequence | 67.9 | 67.9 |
| DQ336086.1 | HIV-1 isolate 3N1T from France LTR, partial sequence | 67.9 | 67.9 |
| DQ336085.1 | HIV-1 isolate X-A2 from France LTR, partial sequence | 67.9 | 67.9 |
| DQ336084.1 | HIV-1 isolate 2N2T from France LTR, partial sequence | 67.9 | 67.9 |
| DQ336083.1 | HIV-1 isolate IX-C1 from France LTR, partial | 67.9 | 104 |
| | sequence | | |
| DQ336082.1 | HIV-1 isolate IX-B5 from France LTR, partial | 67.9 | 67.9 |
| | sequence | | |
| DQ336081.1 | HIV-1 isolate I-B2 from France LTR, partial sequence | 67.9 | 104 |
| DQ336080.1 | HIV-1 isolate B2 from France LTR, partial sequence | 67.9 | 104 |
| DQ336079.1 | HIV-1 isolate A2 from France LTR, partial seq. | 67.9 | 67.9 |
| DQ336078.1 | HIV-1 isolate 3N2T from France LTR, partial | 67.9 | 67.9 |
| | sequence | | |
| DQ336077.1 | HIV-1 isolate VIII-B2 from France LTR, partial | 67.9 | 104 |
| | sequence | | |
| DQ336076.1 | HIV-1 isolate VIII-A2 from France LTR, partial | 67.9 | 104 |
| | sequence | | |
| DQ336075.1 | HIV-1 isolate I-B4 from France LTR, partial sequence | 67.9 | 140 |
| DQ336074.1 | HIV-1 isolate 2N3T from France LTR, partial | 67.9 | 140 |
| | sequence | | |
| DQ336073.1 | HIV-1 isolate 3N3T from France LTR, partial | 67.9 | 104 |
| | sequence | | |
| AM076883.1 | Human immunodeficiency virus 1 proviral 5′ LTR, | 67.9 | 67.9 |
| | TAR element and U3, U5 and R repeat regions, | | |
| | clone PG177.11 | | |
| AM076882.1 | Human immunodeficiency virus 1 proviral 5′ LTR, | 67.9 | 67.9 |
| | TAR element and U3, U5 and R repeat regions, | | |
| | clone PG177.1 | | |
| AM076865.1 | Human immunodeficiency virus 1 proviral 5′ LTR, | 67.9 | 67.9 |
| | TAR element and U3, U5 and R repeat regions, | | |
| | clone PG189.65 | | |
| DQ676879.1 | HIV-1 isolate PS2016_Day380 from Australia, | 67.9 | 67.9 |
| | complete genome | | |
| AB253432.1 | Human immunodeficiency virus 1 proviral DNA, | 67.9 | 135 |
| | complete genome, clone: pBa-L | | |
| AB253431.1 | Human immunodeficiency virus 1 proviral DNA, | 67.9 | 135 |
| | complete genome, clone: pJPDR0769BF6 | | |
| AB253430.1 | Human immunodeficiency virus 1 proviral DNA, | 67.9 | 135 |
| | complete genome, clone: pJPDR0769BF3 | | |
| DQ848523.1 | HIV-1 clone ig20 LTR, partial sequence | 67.9 | 67.9 |
| DQ848522.1 | HIV-1 clone ig19 LTR, partial sequence | 67.9 | 67.9 |
| DQ848521.1 | HIV-1 clone ig18 LTR, partial sequence | 67.9 | 67.9 |
| DQ848520.1 | HIV-1 clone ig17 LTR, partial sequence | 67.9 | 67.9 |
| DQ848518.1 | HIV-1 clone ig15 LTR, partial sequence | 67.9 | 67.9 |
| DQ848517.1 | HIV-1 clone ig14 LTR, partial sequence | 67.9 | 67.9 |
| DQ848516.1 | HIV-1 clone ig13 LTR, partial sequence | 67.9 | 67.9 |
| DQ848515.1 | HIV-1 clone ig12 LTR, partial sequence | 67.9 | 67.9 |
| DQ848514.1 | HIV-1 clone ig11 LTR, partial sequence | 67.9 | 67.9 |
| DQ848513.1 | HIV-1 clone ig10 LTR, partial sequence | 67.9 | 67.9 |
| DQ848512.1 | HIV-1 clone ig9 LTR, partial sequence | 67.9 | 67.9 |
| DQ848511.1 | HIV-1 clone ig8 LTR, partial sequence | 67.9 | 67.9 |
| DQ848509.1 | HIV-1 clone ig6 LTR, partial sequence | 67.9 | 67.9 |
| DQ848508.1 | HIV-1 clone ig5 LTR, partial sequence | 67.9 | 67.9 |
| DQ848507.1 | HIV-1 clone ig4 LTR, partial sequence | 67.9 | 67.9 |
| DQ848506.1 | HIV-1 clone ig3 LTR, partial sequence | 67.9 | 67.9 |
| DQ848505.1 | HIV-1 clone ig2 LTR, partial sequence | 67.9 | 67.9 |
| DQ848504.1 | HIV-1 clone ig1 LTR, partial sequence | 67.9 | 67.9 |
| DQ848503.1 | HIV-1 clone mg12 LTR, partial sequence | 67.9 | 67.9 |
| DQ848502.1 | HIV-1 clone mg11 LTR, partial sequence | 67.9 | 67.9 |
| DQ848501.1 | HIV-1 clone mg10 LTR, partial sequence | 67.9 | 67.9 |
| DQ848500.1 | HIV-1 clone mg9 LTR, partial sequence | 67.9 | 67.9 |
| DQ848499.1 | HIV-1 clone mg8 LTR, partial sequence | 67.9 | 67.9 |
| DQ848498.1 | HIV-1 clone mg7 LTR, partial sequence | 67.9 | 67.9 |
| DQ848497.1 | HIV-1 clone mg6 LTR, partial sequence | 67.9 | 67.9 |
| DQ848496.1 | HIV-1 clone mg5 LTR, partial sequence | 67.9 | 67.9 |
| DQ848495.1 | HIV-1 clone mg4 LTR, partial sequence | 67.9 | 67.9 |
| DQ848494.1 | HIV-1 clone mg3 LTR, partial sequence | 67.9 | 67.9 |
| DQ848493.1 | HIV-1 clone mg2 LTR, partial sequence | 67.9 | 67.9 |
| DQ848492.1 | HIV-1 clone mg1 LTR, partial sequence | 67.9 | 67.9 |
| DQ848491.1 | HIV-1 clone ie26 LTR, partial sequence | 67.9 | 67.9 |
| DQ848485.1 | HIV-1 clone ie9 LTR, partial sequence | 67.9 | 67.9 |
| DQ848483.1 | HIV-1 clone ie7 LTR, partial sequence | 67.9 | 67.9 |
| DQ848478.1 | HIV-1 clone ie1 LTR, partial sequence | 67.9 | 67.9 |
| DQ848474.1 | HIV-1 clone me8 LTR, partial sequence | 67.9 | 67.9 |
| DQ848472.1 | HIV-1 clone me6 LTR, partial sequence | 67.9 | 67.9 |
| DQ848471.1 | HIV-1 clone me5 LTR, partial sequence | 67.9 | 67.9 |
| DQ848467.1 | HIV-1 clone me1 LTR, partial sequence | 67.9 | 67.9 |
| DQ848434.1 | HIV-1 clone md10 LTR, partial sequence | 67.9 | 67.9 |
| DQ848427.1 | HIV-1 clone md3 LTR, partial sequence | 67.9 | 67.9 |
| DQ848426.1 | HIV-1 clone md2 LTR, partial sequence | 67.9 | 67.9 |
| DQ848386.1 | HIV-1 clone ib25 LTR, partial sequence | 67.9 | 67.9 |
| DQ848385.1 | HIV-1 clone ib24 LTR, partial sequence | 67.9 | 67.9 |
| DQ848383.1 | HIV-1 clone ib21 LTR, partial sequence | 67.9 | 67.9 |
| DQ848382.1 | HIV-1 clone ib20 LTR, partial sequence | 67.9 | 67.9 |
| DQ848380.1 | HIV-1 clone ib18 LTR, partial sequence | 67.9 | 67.9 |
| DQ848379.1 | HIV-1 clone ib17 LTR, partial sequence | 67.9 | 67.9 |
| DQ848377.1 | HIV-1 clone ib15 LTR, partial sequence | 67.9 | 67.9 |
| DQ848375.1 | HIV-1 clone ib13 LTR, partial sequence | 67.9 | 67.9 |
| DQ848373.1 | HIV-1 clone ib10 LTR, partial sequence | 67.9 | 67.9 |
| DQ848371.1 | HIV-1 clone ib8 LTR, partial sequence | 67.9 | 67.9 |
| DQ848370.1 | HIV-1 clone ib7 LTR, partial sequence | 67.9 | 67.9 |
| DQ848369.1 | HIV-1 clone ib11 LTR, partial sequence | 67.9 | 67.9 |
| DQ848368.1 | HIV-1 clone ib6 LTR, partial sequence | 67.9 | 67.9 |
| DQ848366.1 | HIV-1 clone ib4 LTR, partial sequence | 67.9 | 67.9 |
| DQ848365.1 | HIV-1 clone ib3 LTR, partial sequence | 67.9 | 67.9 |
| DQ848363.1 | HIV-1 clone ib1 LTR, partial sequence | 67.9 | 67.9 |
| DQ837381.1 | HIV-1 isolate 05CSR3 from South Korea, | 67.9 | 67.9 |
| | complete genome | | |
| DQ672625.1 | HIV-1 clone 1 from Italy envelope glycoprotein | 67.9 | 67.9 |
| | (env) gene, partial cds; and nonfunctional | | |
| | nef protein (nef) gene, complete sequence | | |
| DQ672624.1 | HIV-1 clone 27 from Italy envelope glycoprotein | 67.9 | 67.9 |
| | (env) gene, partial cds; and nonfunctional nef | | |
| | protein (nef) gene, complete sequence | | |
| DQ672623.1 | HIV-1 isolate SG1 from Italy, partial genome | 67.9 | 135 |
| DQ358807.1 | HIV-1 isolate 02BR006 from Brazil, | 67.9 | 67.9 |
| | complete genome | | |
| DQ487191.1 | HIV-1 isolate WCM32P0896 from USA, | 67.9 | 135 |
| | complete genome | | |
| DQ487190.1 | HIV-1 isolate WCM32P0793 from USA, | 67.9 | 135 |
| | complete genome | | |
| DQ487188.1 | HIV-1 isolate WCD32P0793 from USA, | 67.9 | 135 |
| | complete genome | | |
| AB221005.1 | Human immunodeficiency virus 1 proviral DNA, | 67.9 | 135 |
| | complete genome, strain: Ba-L | | |

In each case, query coverage was 100%, E-value was 1e−09, and max. identity was 100%.

According to another aspect the present invention provides a system useful for the detection and isolation of recombinase capable of catalyzing a recombination between two sequence-specific recombinase target sites, the system comprising a plurality of host cells, each cell comprising:

(a) a first nucleic acid construct comprising a transcribable polynucleotide sequence encoding one recombinase of a plurality of recombinases; and

b) a second nucleic acid construct comprising a DNA segment having a 5′ end and a 3′ end wherein the DNA segment is flanked by a pair of sequence-specific recombinase target sites, one at the 5′ end and one at the 3′ end, and wherein the recombinase target site comprises a nucleic acid sequence as set forth in any one of SEQ ID NO:1, SEQ ID NO:2, SEQ ID NO:3 and combinations thereof.

According to certain embodiments, the recombinase target site has a nucleic acid sequence as set forth in any one of SEQ ID NO:4, SEQ ID NO:5, SEQ ID NO:6, SEQ ID NO:7 and SEQ ID NO:8.

According to certain currently preferred embodiments, the recombinase target site is of a nucleic acid sequence as set forth in SEQ ID NO:4.

Alternatively, the second nucleic acid construct of the system of the present invention comprises a recombinase target site having a nucleic acid sequence selected from the group consisting of SEQ ID NO:9 and SEQ ID NO:10.

According to a further aspect, the present invention provides a method of screening for a recombinase enzyme capable of catalyzing a recombination between two recombinase target sites, comprising the steps of:
a) providing a plurality of host cells, each cell comprising:
   i) a first nucleic acid construct comprising a transcribable first polynucleotide sequence encoding one recombinase of a plurality of recombinases; and
   ii) a second nucleic acid construct comprising a DNA segment having a 5′ end and a 3′ end wherein the DNA segment is flanked by a pair of sequence-specific recombinase target sites, one at the 5′ end and one at the 3′ end, and wherein the recombinase target site comprises a nucleic acid sequence as set forth in any one of SEQ ID NO:1, SEQ ID NO:2, SEQ ID NO:3 and combinations thereof;
b) providing suitable conditions such that the plurality of recombinases is expressed in the plurality of host cells;
c) isolating the first and the second nucleic acid constructs from each of said host cells;
d) subjecting the isolated nucleic acid constructs to an assay suitable for detecting excision of said DNA segment from the second nucleic acid construct;
e) selecting a host cell comprising a polynucleotide sequence encoding a recombinase capable of catalyzing the excision of said DNA segment from said second nucleic acid construct; and
f) purifying the recombinase capable of catalyzing said excision or the polynucleotide encoding same.

According to certain embodiments, the recombinase target site has a nucleic acid sequence as set forth in any one of SEQ ID NO:4, SEQ ID NO:5, SEQ ID NO:6, SEQ ID NO:7 and SEQ ID NO:8.

According to certain currently preferred embodiments, the recombinase target site is of a nucleic acid sequence as set forth in SEQ ID NO:4.

According to another aspect, the method of the present invention employs a plurality of host cells, each cell comprising:

i) a first nucleic acid construct comprising a transcribable first polynucleotide sequence encoding one recombinase of a plurality of recombinases; and

ii) a second nucleic acid construct comprising a DNA segment having a 5′ end and a 3′ end wherein the DNA segment is flanked by a pair of sequence-specific recombinase target sites, one at the 5′ end and one at the 3′ end, and wherein the recombinase target site comprises a nucleic acid sequence as set forth in any one of SEQ ID NO:9 and SEQ ID NO:10.

It has been shown that expression of Cre recombinase in cells infected with recombinant HIV into which the wild type loxP sites have been introduced substantially reduced virus replication compared to control cells infected with wild type HIV (Flowers et al. 1997, supra). The mechanism of Cre-mediated inhibition is most likely an excision of proviral DNA from cellular DNA in chromatin. Thus, without wishing to be bound to any particular theory or mechanism of action, recombinases identified using the systems and methods of the present invention are capable of catalyzing excision of HIV-1 proviral DNA in cell infected with the virus, and thus preventing virus replication.

Isolating the nucleic acid constructs from the host cell can be performed by any method known to a person skilled in the art.

For the selection of new recombinase mutants that recognizes the target site and catalyzes recombination event between two such sites, DNA constructs comprising polynucleotides encoding different recombinase mutants should be transformed into cells comprising a DNA construct with a template for the recombinase activity, i.e. a DNA segments flanked by the target site sequences of the present invention or parts thereof. According to certain currently preferred embodiments, the recombinase mutants are Cre mutants.

Any method for the production of recombinase mutant library as is known in the art can be used according to the teaching of the present invention. As exemplified hereinbelow, the wild type Cre gene was mutagenized employing three different approaches: arbitrary substitutions of amino acids along the entire coding region of Cre made by error-prone PCR; site directed mutations along the coding region of Cre, based on crystal structure of the amino acids involved in the DNA interaction between cre and loxP; and rational design of Cre by computer analysis of Cre-loxP interaction as well as the interaction with loxLTR in HIV1 genome.

The DNA constructs of the present invention are preferably encompassed within an expression vector. The recombinant expression vector may optionally include an affinity tag for selection and isolation of protein product encoded by same. Examples of such an affinity tag include, but are not limited to, a polyhistidine tract, polyarginine, glutathione-S-transferase (GST), maltose binding protein (MBP), a portion of staphylococcal protein A (SPA), and various immunoaffinity tags (e.g. protein A) and epitope tags such as those recognized by the EE (Glu-Glu) antipeptide antibodies. The affinity tag may also be a signal peptide either native or heterologous to baculovirus, such as honeybee mellitin signal peptide. The affinity tag may be positioned at either the amino- or carboxy-terminus of the donor DNA. The constructs may also include at least one polynucleotide encoding an antibiotic resistant gene, as a selection marker.

The system of the present invention may comprise a first and a second DNA constructs comprising a polynucleotide encoding a mutated recombinase and a polynucleotide serving as a template for the recombinase activity, respectively, or a combined DNA construct comprising both the recombinase-encoding polynucleotide and its template. In the first case, co-transformation of the two DNA constructs into a single host cell is required for a recombination event to occur.

The system of the present invention may comprise a first a second and a third DNA constructs comprising a polynucleotide encoding a mutated recombinase recognizing SEQ ID NO:1, a polynucleotide encoding a mutated recombinase recognizing SEQ ID NO:3, and a polynucleotide serving as a template for the recombinase activity, respectively, or a combined DNA construct comprising both the two recombinase-encoding polynucleotides and their template. In the first case, co-transformation of the three DNA constructs into a single host cell is required for a recombination event to occur.

The constructs may further comprise a promoter sequence that controls the expression of the recombinase. The promoter may be any array of DNA sequences that interact specifically with cellular transcription factors to regulate transcription of the downstream gene. The promoter may be derived from any organism, such as bacteria, yeast, insect and mammalian cells and viruses. The selection of a particular promoter depends on what cell type is to be used to express the protein of interest.

The constructs of the present invention further comprise specific nucleic acid tags for identifying recombination events, specifically, excision of a certain DNA segment from the construct. Such tags may include specific nucleic acid sequences serving as templates for DNA amplification using compatible primes, enzyme-specific restriction sequences, polynucleotides encoding polypeptides capable of conferring antibiotic resistance which are expressed only after a recombination event has been occurred and the like, as is known to a person skilled in the art.

Other than containing the necessary elements for the transcription and translation of the inserted coding sequence, the expression constructs or vectors of the present invention can also include sequences engineered to enhance stability, production, purification, yield or toxicity of the expressed recombinases. For example, the expression of a fusion protein comprising the recombinase and a heterologous protein can be engineered. With such design the recombinase can be readily isolated by affinity chromatography; e.g., by immobilization on a column specific for the heterologous protein. Where a cleavage site is engineered between the recombinase moiety and the heterologous protein, the recombinase can be released from the chromatographic column by treatment with an appropriate enzyme or agent that disrupts the cleavage site (e.g., see Booth et al. (1988) Immunol. Lett. 19: 65-70; and Gardella et al., (1990) J. Biol. Chem. 265: 15854-15859).

A variety of prokaryotic or eukaryotic cells can be used as host-expression systems to express the recombinase coding sequence. These include, but are not limited to, microorganisms, such as bacteria transformed with a recombinant bacteriophage DNA, plasmid DNA or cosmid DNA expression vector containing the recombinase coding sequence; yeast transformed with recombinant yeast expression vectors containing the recombinase coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the recombinase coding sequence. Mammalian expression systems can also be used to express recombinase. Bacterial systems are preferably used to produce recombinant recombinase, according to the present invention, thereby enabling a high production volume at low cost.

Cells transformed with the DNA construct expressing recombinase are cultured under effective conditions, which allow for the expression of recombinase at the amount required for catalyzing the recombination between the LTR4 target sites. Effective culture conditions include, but are not limited to, effective media, bioreactor, temperature, pH and oxygen conditions that permit protein production. An effective medium refers to any medium in which a cell is cultured to produce the plurality of mutated recombinase and enable its activity. Such a medium typically includes an aqueous solution having assimilable carbon, nitrogen and phosphate sources, and appropriate salts, minerals, metals and other nutrients, such as vitamins. Host cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Isolation of nucleic acid from the library of host cells of the present invention can be done by any methods as is known to a person skilled in the art. The isolated nucleic acids are then screened to identify recombination events, specifically excision events, employing the suitable means according to the nucleic acid tag incorporated to the DNA construct. Typically, the isolated nucleic acids are subjected to restriction by specific enzymes, PCR reactions and combinations thereof. According to currently certain preferred embodiments, the recombination event confers antibiotic resistance on the host cell, such that the occurrence of a recombination event is detected by the ability of the host cell to grow in a medium containing the antibiotic.

Once a clone is identified in a screen such as the one described above, it can be isolated or plaque purified and sequenced. The insert may then be used in other cloning reactions, for example, cloning into an expression vector that enables efficient production of the recombinase.

Depending on the vector and host system used for production, resultant proteins may either remain within the recombinant cell; be secreted into the fermentation medium; be secreted into a space between two cellular membranes, such as the periplasmic space in E. coli; or be retained on the outer surface of a cell or viral membrane.

Following culturing, recovery of the recombinant enzyme is performed. The phrase “recovering the recombinant enzyme” refers to collecting the whole fermentation medium containing the protein and need not imply additional steps of separation or purification. Recombinases identified by the system and method of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Expression determination of the hereinabove described recombinant proteins can be affected using specific antibodies, which recognize the recombinases identified using the system and methods of the present invention. Aside from their important usage in detection of expression of a recombinase, these antibodies can be used as to screen expression libraries and/or to recover desired recombinase enzymes from a mixture of proteins and other contaminants.

### EXAMPLES

### Example 1

### Activity of Wild Type Cre Enzyme on lox-LTR Variant Sequences

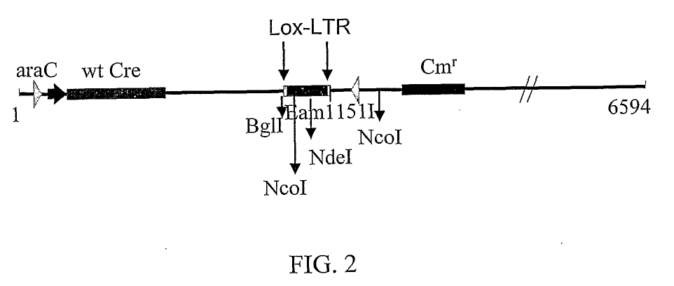

Cre enzyme activity was tested using derivatives of the vector pBAD-33 (SEQ ID NO:30; Buchholz and Stewart (2001) Nat. Biotechnol. 19: 1047-1052, 2001). A schematic representation of derivatives of pBAD-33 is shown in FIG. 2. The vector contains two loxP-type sites and can be used for screening to identify Cre variants that recognize the sites. Between the two loxP sites is a 294 bp fragment that contains one of the two NcoI sites and the unique NdeI site of the vector. Cre-mediated recombination thus generates a circular product that is not digested by NdeI and is linearized by NcoI (or NdeI/NcoI digestion), yielding a 6440 bp fragment. PCR amplification of the lox-containing region of a recombined plasmind yields a 1950 bp PCR product. If the pBAD-33 vector has not undergone recombination, digestion with NcoI+NdeI gives rise to 5440 bp and 1300 bp fragments. PCR of the undigested, unrecombined plasmid yields a 2244 bp PCR product; prior NdeI/NcoI digestion prevents formation of this product.

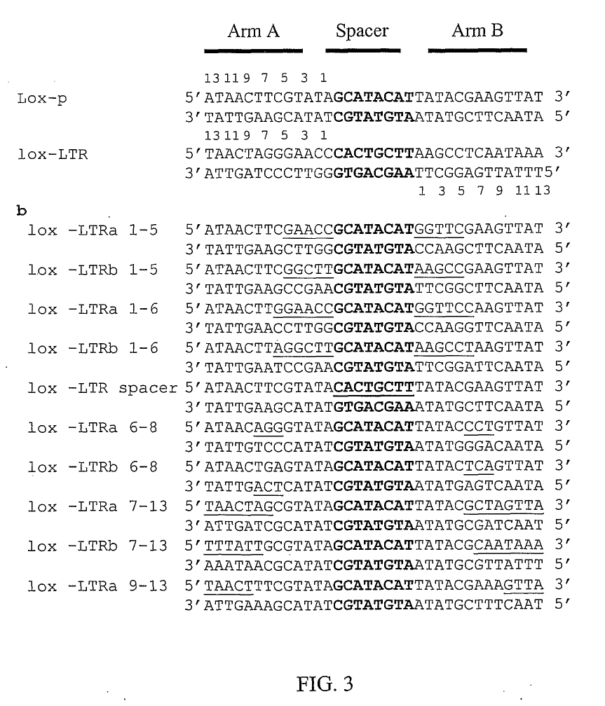

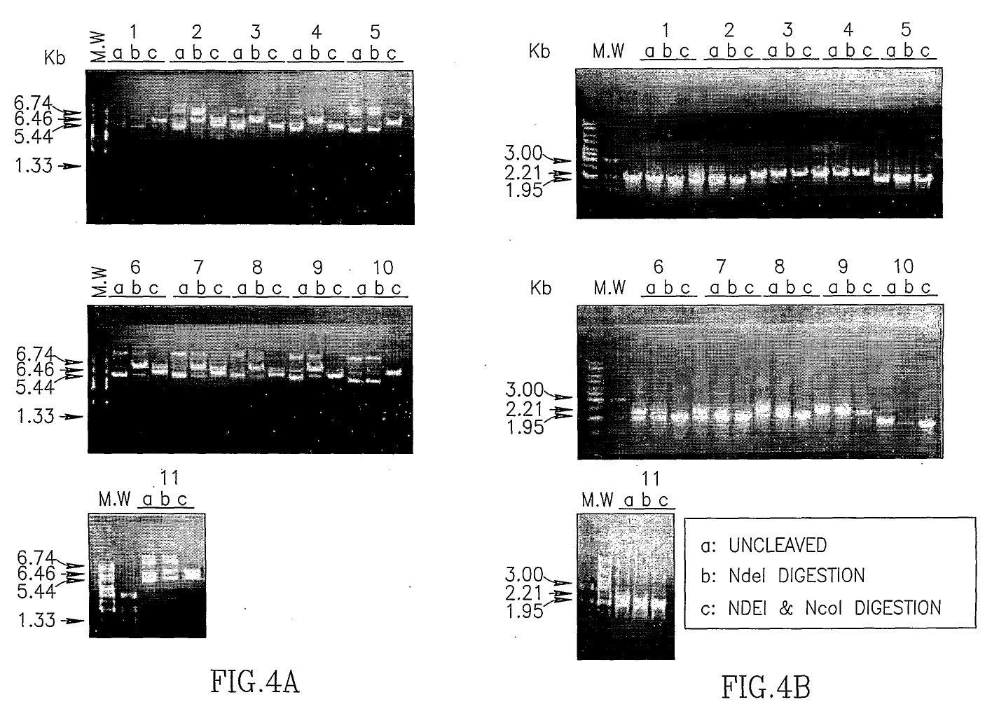

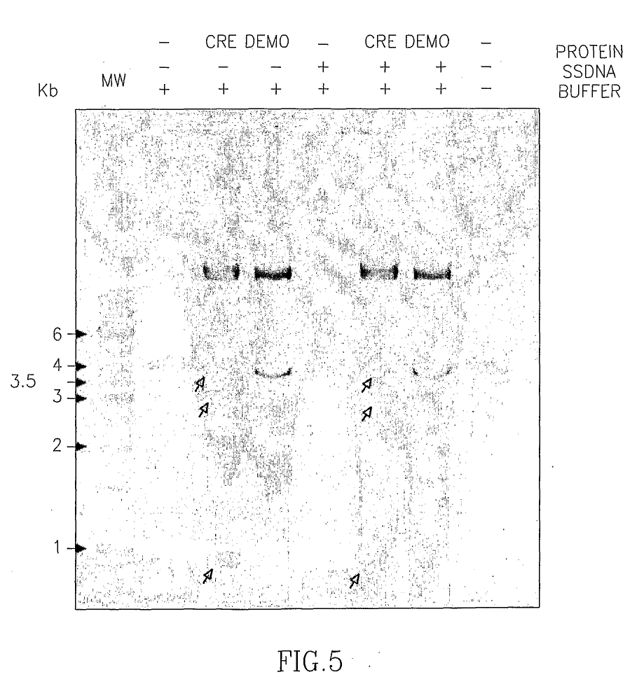

Eleven mutated versions of the loxP-like substrate found in the LTR (FIG. 3) were cloned into pBAD-33 and analyzed for recombination in the presence of wild-type (wt) Cre. After transformation and growth under conditions allowing the expression of the Cre variants, plasmid vectors were harvested and subjected to restriction enzyme digest alone (A) or digest+PCR analysis (B), in order to amplify the loxP-containing fragment and fractionated on an agarose gel (FIG. 4B).

In the case of a positive control DNA construct comprising the loxP inverted repeats, the expressed wt recombinase excised the DNA fragment between the loxP sites, eliminating the intervening NdeI and NcoI sites. As expected, this vector was linearized by NcoI digestion, yielding a 6440 bp fragment (FIG. 4A lane 1C). PCR amplification of the product of the loxP control produced a 1950 bp DNA fragment, demonstrating 100% excision activity by wt Cre (FIG. 4B, lanes 1A-C). An activity approximately equal to the wild-type loxP sites was observed with wt Cre and the mutated lox sites lox-LTRa 9-13 and lox-LTRb 7-13 substrates (FIG. 4A-B lanes 5A-C and lanes 10A-C).

Partial recombination was obtained with the following substrates: lox-LTR-spacer, lox-LTRa6-8, lox-LTRa7-13, lox-LTRb 1-5 and lox-LTRb 6-8, as indicated by the presence of a mixture of linearized fragment (size 6740 bp) and circular non-cleaved recombination product following digestion with NdeI (FIG. 4A, lanes A-C of 2, 6, 7, 8, and 11). PCR amplification of these samples yielded a mixture of 1950 bp and 2250 bp in the uncleaved forms (FIG. 4B lanes A of 2, 6, 7, 8, and 11)) and in all cleaved forms resulted with the recombinant fragment size of 1950 bp (FIG. 4B lanes B and C of 2, 6, 7, 8, and 11).

Plasmids for which no recombination event occurred, namely LTRa 1-5, LTRa 1-6, and LTRb1-6, generated 5440 bp and 1300 bp fragments upon double digestion with NdeI and NcoI (FIG. 4A, lane C of 3, 4, and 9). Digestion with NdeI linearized this plasmid to 6740 bp (FIG. 4A lane B of 3, 4, and 9). In these constructs, no PCR amplification was expected following single or double digestion. However, PCR fragments of 2250 bp were observed due to incomplete digestion (FIG. 4B lanes A-C of 3, 4, and 9).

Several reports demonstrated that the essential interaction between Cre recombinase and loxP substrate is located in the nucleotide base pairs closer to positions 1-7 of the spacer and that changing these nucleotides has a major effect on the recombination catalysis. Changing the nucleotide in position 8-13 has a rather minor effect on the interaction (Hartung and Kisters-Woike (1998) J. Biol. Chem., 273: 22884-22891). The crystal structure of the Cre-loxP supports this assumption (Guo, et al., (1997) Nature, 389: 40-46). The tolerance of wt Cre for changes in the 8 by spacer region has been shown (Lee and Saito (1998) Gene 216: 55-65). Wt Cre facilitate efficiently recombination of lox-LTRb 7-13, wherein position 7 has changed to G instead of T, while such a change has a very minor effect on Cre catalysis (Hartung and Kisters-Woike 1998, supra). Nucleotides 8-9 and 12 in lox-LTRb 7-13 are identical to loxP; therefore lox-LTRb 7-13 differs only slightly in the recombination efficiency compared to loxPy. Lox-LTRa 7-13 also comprises a change to G at position 7; however, a reduction of recombination was observed. This may be due to the different combinations of nucleotides at positions 8-10 and 12-13, which disrupt and weakened the interaction between wt Cre and lox-LTRa7-13.

### Example 2

### Identification of Cre Variants Recognizing Novel Cre Sites

### Materials and Experimental Methods

Library Production

Three different approaches were taken for mutating the gene encoding wild-type Cre recombinase:

1. Arbitrary substitutions of amino acids along the entire coding region of Cre, generated by error-prone PCR.

2. Site-directed mutagenesis along the coding region of Cre of 50 amino acids involved in the DNA interaction between Cre and lox-P, based on the crystal structure. The library was constructed to enable all possible amino acid substitutions within the 50 amino acids sites, with a 50% chance for a substitution at each single amino acid. This method is achieved using degenerate primers containing NNK codon. In each case, the N nucleotide mixture contained 70% restoration of the original nucleotide and 10% for each of the other 3 nucleotides. The K nucleotide mixture contained 50% each of guanine and thymine nucleotides.

3. Rational design of Cre by computer analysis of Cre-loxP interaction as well as the interaction with loxP-like LTR sequences in HIV1 genome.

An error-prone library was constructed as follows: The open reading frame of wild-type Cre (GI:15135) was cloned to pGEM®T-easy (Promega) using PCR amplification with primers 5′ TCGAGCTCTGTACAAGGAGGAATTCACCATGTCCAATTTACTGACCGTAC 3′(SEQ ID NO:24) containing Sad and BsrGI restriction sites and 5′ CTCTAGACTAATCGCCATCTTCCAGCAGG 3′(SEQ ID NO:25) containing XbaI restriction site. This plasmid was used as a template DNA for PCR manipulation with Mutazyme™ II DNA polymerase using the GeneMorph™ random mutagenesis kit (Stratagene). The first round of error-prone PCR was performed with primers 5′CTTCGCTATTACGCCAGCTGGC 3′ (SEQ ID NO:26) and 5′CACTTTATGCTTCCGGCTCG 3′ (SEQ ID NO:27). The amplified fragment, having a size of 1524 bp was extracted from agarose gel and 20 ng of the product was subjected to a second round of error-prone PCR using the commercial primers T7 and SP6-pGEM®-T as nested primers. The PCR fragments having a size of 1200 bp were extracted and cut with the restriction enzymes BsrGI and XbaI and ligated into pBAD-33 recombinant vector (purchased from ATCC) cut with the same enzymes. The pGEM®T-easy containing wild type Cre was cut with Sad and XbaI and ligated to pBAD33 cut by the same restriction sites Sad and XbaI at positions 4668 and 4689, respectively.

The feature allowing selection of an active recombinase is that the distance between the ampicillin (Amp) promoter and the Amp-resistance gene is too large and such that the gene is not activated by its promoter. Thus, bacteria containing the non-recombinant plasmid are sensitive to ampicillin and do not grow in media containing ampicillin. In the presence of an active recombinase that excises the DNA fragment flanked by the two recombinase sites, the promoter becomes operably linked to the Amp resistance gene and activates its transcription. Accordingly, bacteria containing the adequate recombinase enzyme are resistant to ampicillin.

### Library Transformation

The library was ligated into the selection plasmid followed by transformation into bacteria. During the transformation process, bacteria were grown in the presence of arabinose for 2 hours to induce Cre expression. The bacteria were grown in liquid culture containing chloramphenicol but lacking ampicillin to select for bacteria that contain a plasmid. Glucose was also added to the culture medium to shut down completely the Ara promoter. A small portion was plated to determine library complexity (efficiency of transformation).

### Plasmid Preparation

The bacterial culture was harvested and a plasmid preparation was made. The plasmid preparation includes all plasmids, of which only a small portion is expected to be recombinant.

### Selection of Recombinant Plasmids

About 100 ng of the plasmid was transformed into bacteria. In parallel, the same amount of the control plasmid, containing wild-type Cre, was transformed. The resulting bacteria are plated on Amp (plus Chl and Glucose) with a small amount plated on Chl (plus glucose) to determine transformation efficiency. As the background of the bacteria is 1 colony per 10⁶, only a modest increase in the library over the control is expected.

### Sub-Cloning of Selected Cre Variants

The colonies obtained from the previous stage (Amp resistant from the library) were all collected and grown together. Plasmid was prepared from the resulting culture, and Cre was excised and sub-cloned into the original selection plasmid.

### Final Transformation

As above, 100 ng were used to transform bacteria and a similar 100 ng from the control were used. Again, the bacteria were plated on Amp (plus Chl and Glucose) with a small amount plated on Chl (plus glucose) to determine transformation efficiency. Enriching for Cre variants recognizing the LTR4 site was expected, which is reflected in a high difference between the experimental and control groups.

### Results

Directed evolution of wild-type Cre recombinase was used to identify Cre variants that recognize target sites of interest. The system was initially tested on wild-type Cre acting on its normal loxP target site, using a plasmid that is shortened by 1000 nucleotides as a consequence of recombination. Following incubation of the plasmid with the protein extract containing the Cre protein, the reaction was examined on agarose gel. A shorter plasmid and a 1000 nucleotide fragment were observed following the incubation with extracts containing the Cre protein (FIG. 5, lanes 3 and 6).

In additional experiments, activity of wild-type Cre was tested with arm-A and arm-B target sequences TAACTAGGGAACCCACTGCTTGGTTCCCTAGTTA (SEQ ID NO:6) and TTTATTGAGGCTTCACTGCTTAAGCCTCAATAAA (SEQ ID NO:8). Substrate plasmids similar to those described in the above paragraph, but containing the arm-A and arm-B target sequences were added to the protein mix and incubated for several hours at 37° C. Strong activity was observed for wild-type Cre with loxP target sites, which served as a positive control. A slight background activity was noted in the control reaction in which no Cre was expressed in the cells (NoCre LTR1-34 lane). Several Cre variants were identified as exhibiting significant recombinase activity on the arm-A and arm-B sequences.

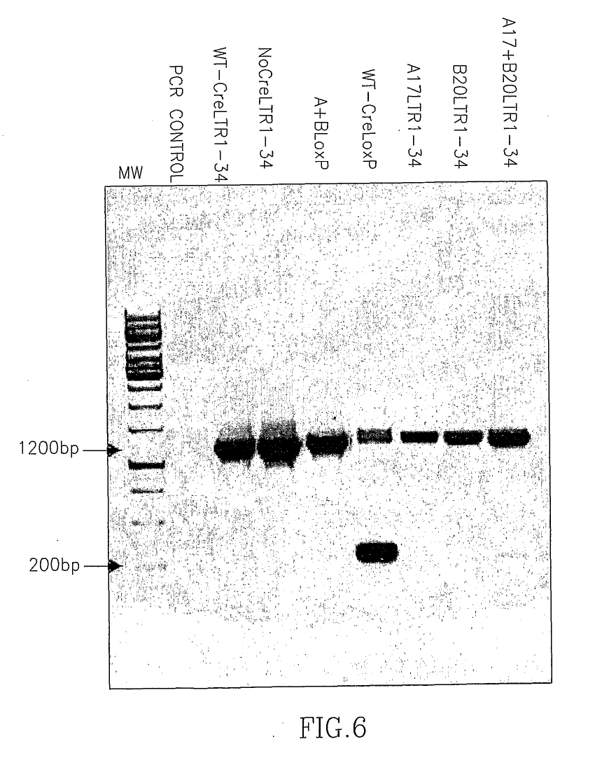

In the next experiment, Cre variants identified in the directed evolution experiment were mixed and activity was assayed in vitro, using the wt loxP-like LTR sequence TAACTAGGGAACCCACTGCTTAAGCCTCAATAAA (SEQ ID NO:4). The mixture of clone A 17 of the A-arm Cre variant and clone B20 of the B-arm Cre variant exhibited higher-than-background activity, which was also significantly higher than A17 alone or B20 alone (FIG. 6).

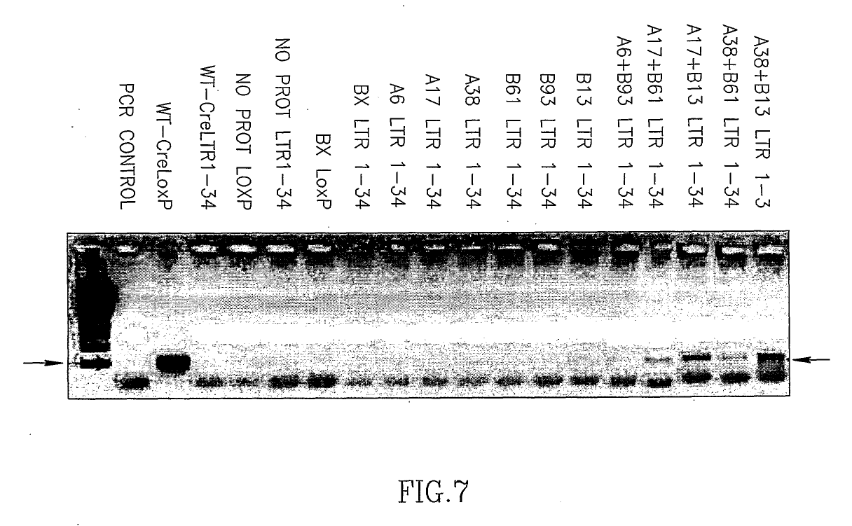

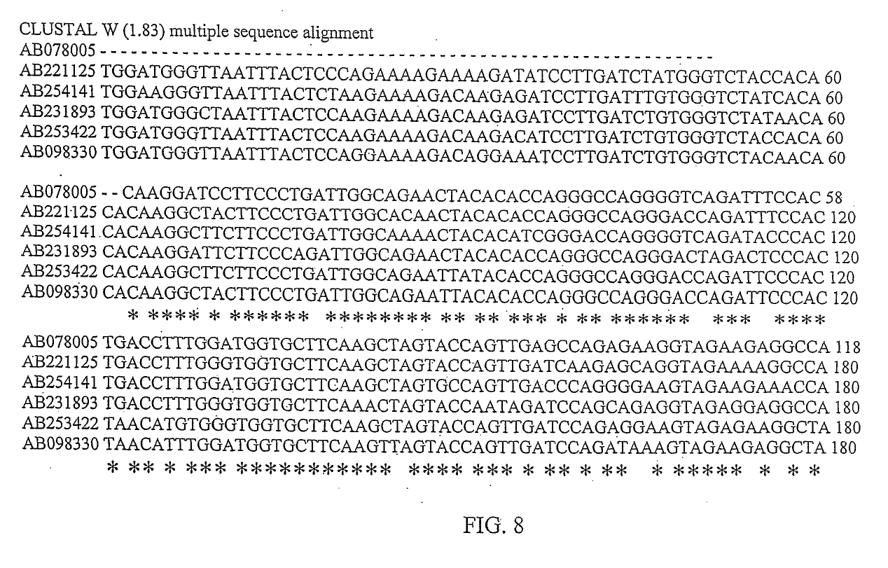

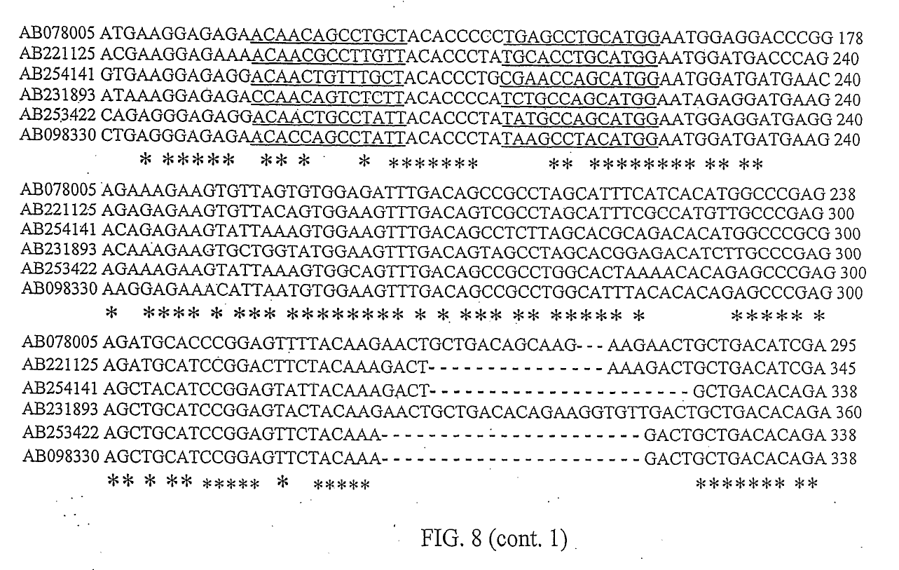

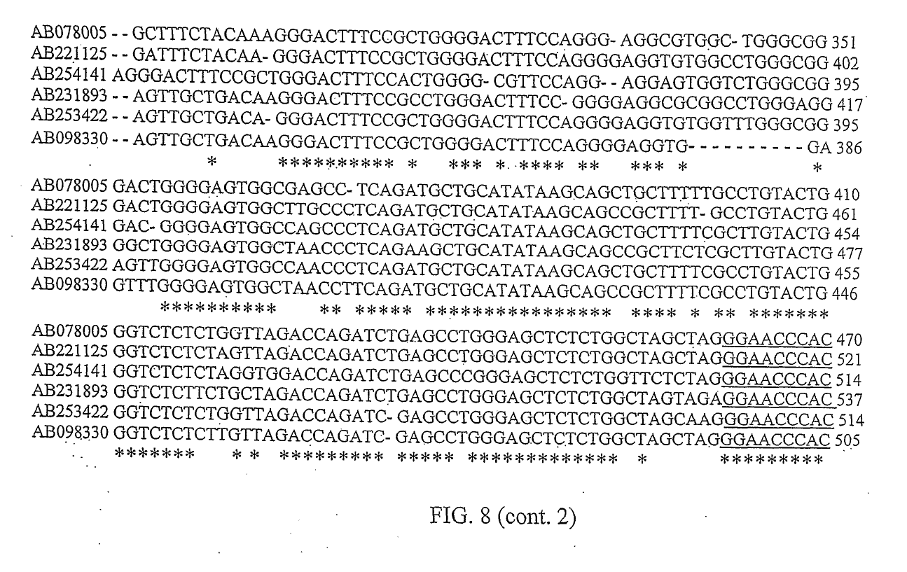

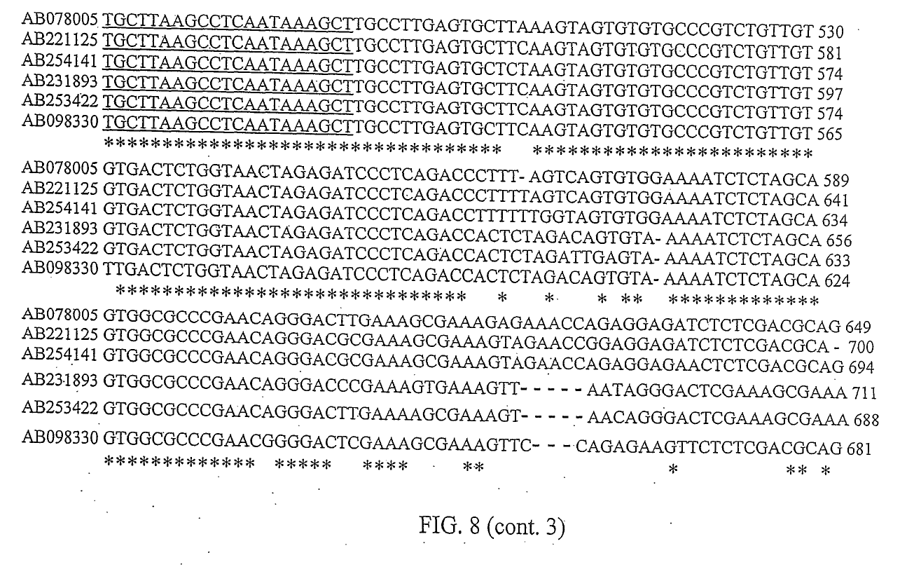

Next, a different in vitro assay was used to test the activity of Cre variants, alone and in combination. Again, strong activity was observed for positive control wild-type Cre with loxP target sites (FIG. 7). In this assay the background activity was undetectable (lanes BX-loxP and BX-LTR1-34). Significant recombination activity on the LTR1-34 substrate was observed for A17+B13 and to a greater extent for A38+B13. Each Cre variant alone except B13 (which exhibited low activity) exhibited no detectable activity on the LTR1-34 substrate.

The above data show that mutant loxP-sequences of the present invention are useful in identification of Cre variant proteins that have great potential in treatment of HIV-1.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A polynucleotide comprising an isolated HIV-1 nucleic acid sequence selected from the group consisting TAACTAGGGAACC (SEQ ID NO:1), CACTGCTT (SEQ ID NO:2) and AAGCCTCAATAAA (SEQ ID NO:3), wherein the length of said isolated HIV-1 nucleic acid sequence is from about 30 to about 100 nucleic acids.

2. The isolated HIV-1 nucleic acid fragment according to claim 1, comprising the nucleic acid sequence TAACTAGGGAACCCACTGCTTAAGCCTCAATAAA (SEQ ID NO:4).

3. A construct comprising the isolated HIV-1 nucleic acid fragment of claim 1.

4. An expression vector comprising the isolated HIV-1 nucleic acid fragment of claim 1.

5. A host cell comprising the expression vector of claim 4.

6. A polynucleotide comprising the nucleic acid sequence TAACTAGGGAACCnnnnnnnnGGTTCCCTAGTTA (SEQ ID NO:12), wherein “nnnnnnnn” represents any combination of nucleic acid bases.

7. The polynucleotide of claim 6, wherein said nucleic acid sequence is selected from the group consisting of TAACTAGGGAACCGCATACATGGTTCCCTAGTTA (SEQ ID NO:5) and TAACTAGGGAACCCACTGCTTGGTTCCCTAGTTA (SEQ ID NO:6).

8. An expression vector comprising the polynucleotide of claim 6.

9. A host cell comprising the expression vector of claim 8.

10. A polynucleotide comprising the nucleic acid sequence TTTATTGAGGCTTnnnnnnnnAAGCCTCAATAAA (SEQ ID NO:13), wherein “nnnnnnnn” represents any combination of nucleic acid bases.

11. The polynucleotide of claim 10, wherein said nucleic acid sequence is selected from the group consisting of TTTATTGAGGCTTGCATACATAAGCCTCAATAAA (SEQ ID NO:7) and TTTATTGAGGCTTCACTGCTTAAGCCTCAATAAA (SEQ ID NO:8).

12. An expression vector comprising the polynucleotide of claim 10.

13. A host cell comprising the expression vector of claim 12.

14. A polynucleotide comprising a nucleic acid sequence selected from the group consisting of TAACTAGGGAACCnnnnnnnnAAGCCTCAATAAA (SEQ ID NO:31), wherein “nnnnnnnn” represents any combination of nucleic acid bases other than CACTGCTT; ACCCACTGCTTAAnnnnnnnnAAAGCTTGCCTTG (SEQ ID NO:14), wherein “nnnnnnnn” represents any combination of nucleic acid bases other than GCCTCAAT or wherein “nnnnnnnn” represents GCCTCAAT (SEQ ID NO:9); and CTGCTTAAGCCTCnnnnnnnnTTGCCTTGAGTGC (SEQ ID NO:15), wherein “nnnnnnnn” represents any combination of nucleic acid bases other than AATAAAGC or wherein “nnnnnnnn” represents AATAAAGC (SEQ ID NO:10).

15. An expression vector comprising the polynucleotide of claim 14.

16. A host cell comprising the expression vector of claim 15.

17-24. (canceled)

25. A kit for measuring the recombinase activity of an enzyme, the kit comprising a plurality of host cells, each cell comprising the polynucleotide of
(a) an isolated HIV-1 nucleic acid sequence selected from the group consisting TAACTAGGGAACC (SEQ ID NO:1), CACTGCTT (SEQ ID NO:2) and AAGCCTCAATAAA (SEQ ID NO:3), wherein the length of said isolated HIV-1 nucleic acid sequence is from about 30 to about 100 nucleic acids;
(b) a nucleic acid sequence TAACTAGGGAACCnnnnnnnnGGTTCCCTAGTTA (SEQ ID NO:12), wherein “nnnnnnnn” represents any combination of nucleic acid bases;
(c) a nucleic acid sequence TTTATTGAGGCTTnnnnnnnnAAGCCTCAATAAA (SEQ ID NO:13), wherein “nnnnnnnn” represents any combination of nucleic acid bases; or
(d) a nucleic acid sequence selected from the group consisting of TAACTAGGGAACCnnnnnnnnAAGCCTCAATAAA (SEQ ID NO:31), wherein “nnnnnnnn” represents any combination of nucleic acid bases other than CACTGCTT; ACCCACTGCTTAAnnnnnnnnAAAGCTTGCCTTG (SEQ ID NO:14), wherein “nnnnnnnn” represents any combination of nucleic acid bases other than GCCTCAAT or wherein “nnnnnnnn” represents GCCTCAAT (SEQ ID NO:9); and CTGCTTAAGCCTCnnnnnnnnTTGCCTTGAGTGC (SEQ ID NO:15), wherein “nnnnnnnn” represents any combination of nucleic acid bases other than AATAAAGC or wherein “nnnnnnnn” represents AATAAAGC (SEQ ID NO:10).

26-30. (canceled)
